(19) United States
(12) Patent Application Publication
(10) Pub. No.: US 2007/0179162 A1
(43) Pub. Date: Aug. 2, 2007

(54) Triazolopyrimdines

(76) Inventors: Olaf Gebauer, Koln (DE); Ulrich Heinemann, Leichlingen (DE); Jorg Nico Greul, Leichlingen (DE); Stefan Herrmann, Langenfeld (DE); Oliver Guth, Leverkusen (DE); Herbert Gayer, Monheim (DE); Hans-Ludwig Elbe, Wuppertal (DE); Stefan Hillebrand, Neuss (DE); Ronald Ebbert, Nurnberg (DE); Ulrike Wachendorff-Neumann, Neuwied (DE); Karl-Heinz- Kuck, Langenfeld (DE)

Correspondence Address: STERNE, KESSLER, GOLDSTEIN & FOX P.L.L.C., 1100 NEW YORK AVENUE, N.W., WASHINGTON, DC, 20005, US

(21) Appl. No.: 10/560,438

(22) PCT Filed: Jun. 14, 2004
(86) PCT No.: PCT/EP04/06368

§ 371 Date: Apr. 25, 2006

(30) Foreign Application Priority Data
Jun. 24, 2003 (DE) 103 28 171.1

(51) Int. Cl. A61K 31/519; C07D 487/04

(52) U.S. Cl. 514/259.31; 544/263

(57) ABSTRACT

The invention relates to novel triazolopyrimidines of formula (I), in which R¹, R², R³ and X are defined as cited in the description, to a method for producing said substances and to their use for controlling undesirable micro-organisms. The invention also relates to novel intermediate products of the formulae (II), (VI), (VII-a) and (VII-b), in addition to methods for producing said substances.

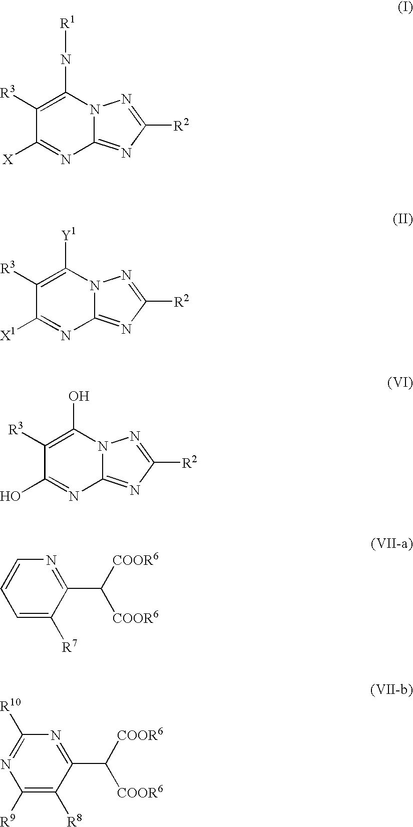

The present invention relates to new triazolopyrimidines, a method for their production, and their use for combating undesired micro-organisms. In addition, the present invention relates to new intermediate products and methods for their production.

It is already known that specific triazolopyrimidines have fungicidal properties (cf. WO 99-41 255, WO 02-02 563, JP-A 2002-308 878, WO 03-04 465 and WO 03-08 417). The efficiency of these substances is good but in some cases, leaves something to be desired when low quantities are used.

New triazolopyrimidines of the formula 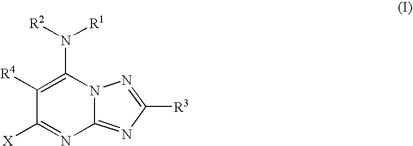
in which
R¹ represents optionally substituted alkyl, optionally substituted alkenyl, optionally substituted alkynyl, optionally substituted cycloalkyl, optionally substituted cycloalkenyl, or optionally substituted heterocyclyl, which is linked via carbon,
R² represents hydrogen, halogen, optionally substituted alkyl, or optionally substituted cycloalkyl,
R³ represents optionally substituted heterocyclyl,
X represents halogen, cyano, optionally substituted alkyl, optionally substituted alkoxy, optionally substituted alkylthio, optionally substituted alkylsulphinyl, or optionally substituted alkylsulphonyl,
   have now been found.

Furthermore, it has been found that triazolopyrimidines of the formula (I) may be produced by reacting
(a) dihalogentriazolopyrimidines of the formula 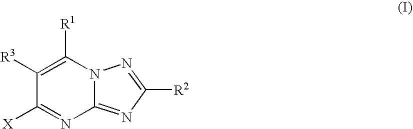
in which
R² and R³ have the meanings specified above,
X¹ represents halogen and
Y¹ represents halogen,
with metal compounds of the formula
R¹-Me (III)
in which
R¹ has the meaning specified above
Me represents lithium, dihydroxyboranyl or a residue of the formula 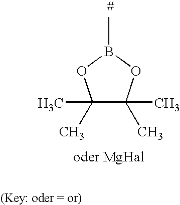
in which
Hal represents chlorine or bromine,
   optionally in the presence of a diluent, optionally in the presence of an acid acceptor, and optionally in the presence of a catalyst, and optionally reacting the triazolopyrimidines of the formula R¹ 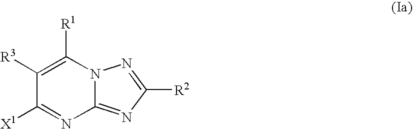
   in which
   R¹, R², R³ and X¹ have the meanings specified above,
   either
   α) with compounds of the formula
   R⁴-Me¹ (TV)
in which
R⁴ represents optionally substituted alkoxy, optionally substituted alkylthio, optionally substituted alkylsulphinyl, optionally substituted alkylsulphonyl, or cyano and
Me¹ represents sodium or potassium,
optionally in the presence of a diluent,
or
   β) with Grignard compounds of the formula
   R⁵—MgHal¹ (V)
in which
R⁵ represents optionally substituted alkyl and
Hal¹ represents chlorine or bromine,
in the presence of a diluent.

Finally, it has been found that triazolopyrimidines of the formula (I) are very well suitable for combating undesired micro-organisms. Above all, they display a strong fungicidal activity and may be used both in plant protection and also in material protection.

Surprisingly, the triazolopyrimidines of the formula (I) have a significantly better microbicidal activity than the most constitutionally similar previously known materials of identical direction of activity.

The compounds of the formula (I) according to the present invention may optionally be provided as mixtures of different possible isomeric forms, particularly stereoisomers, such as E and Z, threo and erythro, and also optical isomers, such as R and S isomers or atropisomers, optionally even tautomers.

Both the pure stereoisomers and also any arbitrary mixtures of these isomers are the object of the present invention, even if generally only the compounds of the formula (I) are discussed here.

Depending on the type of the substituents defined above, the compounds of the formula (I) have acid or basic properties and may form salts. If the compounds of the formula (I) carry hydroxy, carboxy, or other groups which induce acid properties, these compounds may be reacted with bases to produce salts. Suitable bases are, for example, hydroxides, carbonates, hydrogen carbonates of the alkaline and alkaline earth metals, particularly those of sodium, potassium, magnesium, and calcium, as well as ammonia, primary, secondary, and tertiary amines having (C₁-C₄) alkyl residues as well as mono-, di-, and trialkanolamines of (C₁-C₄) alkanols. If the compounds of the formula (I) have amino, alkylamino, or other groups inducing basic properties, these compounds may be reacted with acids to produce salts. Suitable acids are, for example, mineral acids, like hydrochloric acid, sulphuric acid, and phosphoric acid, organic acids such as acetic acid or oxalic acid, and acid salts, such as NaHSO₄ and KHSO₄. The salts which may thus be obtained also have fungicidal and microbicidal properties.

The object of the present invention is also the salt-like derivatives produced from compounds of the formula (I) through reaction with the basic and/or acidic compounds as well as the N oxides producible according to typical oxygenation methods.

In the present case, heterocyclyl represents saturated or unsaturated, aromatic or non-aromatic cyclic compounds having 3 to 8 ring members, in which at least one ring member represents a heteroatom, i.e., an atom different from carbon. If the ring contains multiple heteroatoms, these may be identical or different. Heteroatoms are preferably oxygen, nitrogen, or sulphur. If the ring contains multiple oxygen atoms, these are not directly neighboring. The cyclic compounds optionally jointly form a polycyclic ring system with further carbocyclic or heterocyclic, fused or bridged rings. Monocyclic or bicyclic ring systems, particularly monocyclic or bicyclic aromatic ring systems are preferred.

The triazolopyrimidines according to the present invention are generally defined by the formula (I). Those materials of the formula (I), in which
R¹ represents alkyl having 1 to 6 carbon atoms, which may be substituted one to five times, identically or differently, by halogen, cyano, hydroxy, alkoxy having 1 to 4 carbon atoms, tri(C₁-C₄ alkyl)silyl and/or cycloalkyl having 3 to 6 carbon atoms, which may be substituted one to three times, identically or differently by halogen, halogenalkyl having 1 or 2 carbon atoms and 1 to 5 halogen atoms and/or alkyl having 1 to 4 carbon atoms, or
R¹ represents alkenyl having 2 to 6 carbon atoms, which may be substituted one to three times, identically or differently by halogen, cyano, hydroxy, alkoxy having 1 to 4 carbon atoms, tri(C₁-C₄ alkyl)silyl and/or cycloalkyl having 3 to 6 carbon atoms, which may be substituted one to three times, identically or differently by halogen, halogenalkyl having 1 or 2 carbon atoms and 1 to 5 halogen atoms and/or alkyl having 1 to 4 carbon atoms, or
R¹ represents alkynyl having 3 to 6 carbon atoms, which may be substituted one to three times, identically or differently by halogen, cyano, alkoxy having 1 to 4 carbon atoms, tri(C₁-C₄ alkyl)silyl and/or cycloalkyl having 3 to 6 carbon atoms, which may be substituted one to three times, identically or differently by halogen, halogenalkyl having 1 or 2 carbon atoms and 1 to 5 halogen atoms and/or alkyl having 1 to 4 carbon atoms, or
R¹ represents cycloalkyl having 3 to 6 carbon atoms, which may be substituted one to three times, identically or differently by halogen, halogenalkyl having 1 or 2 carbon atoms and 1 to 5 halogen atoms and/or alkyl having 1 to 4 carbon atoms, or
R¹ represents cycloalkenyl having 3 to 6 carbon atoms, which may be substituted one to three times, identically or differently by halogen and/or alkyl having 1 to 4 carbon atoms, or
R¹ represents saturated or unsaturated heterocyclyl, linked via carbon, having 5 or 6 ring members and 1 to 3 heteroatoms, such as nitrogen, oxygen, and/or sulphur, the heterocyclyl able to be substituted once or twice by halogen, alkyl having 1 to 4 carbon atoms, cyano, nitro, alkoxy having 1 to 4 carbon atoms, cycloalkyl having 3 to 6 carbon atoms, halogenalkyl having 1 to 4 carbon atoms and 1 to 9 halogen atoms, and/or halogenalkoxy having 1 to 4 carbon atoms and 1 to 9 halogen atoms
R² represents hydrogen, fluorine, chlorine, bromine, iodide, alkyl having 1 to 4 carbon atoms, halogenalkyl having 1 to 4 carbon atoms and 1 to 9 halogen atoms, or cycloalkyl having 3 to 6 carbon atoms,
R³ represents saturated or unsaturated heterocyclyl having 5 or 6 ring members and 1 to 4 heteroatoms, such as oxygen, nitrogen and/or sulphur, the heterocyclyl being able to be substituted one to four times, identically or differently by fluorine, chlorine, bromine, cyano, nitro,
   alkyl, alkoxy, hydroximinoalkyl or alkoximinoalkyl each having 1 to 3 carbon atoms per alkyl part,
   halogenalkyl or halogenalkoxy each having 1 to 3 carbon atoms and 1 to 7 halogen atoms,
   and
X represents fluorine, chlorine, bromine, cyano, alkyl having 1 to 4 carbon atoms, alkoxy having 1 to 4 carbon atoms, alkylthio having 1 to 4 carbon atoms, alkylsulphinyl having 1 to 4 carbon atoms, or alkylsulphonyl having 1 to 4 carbon atoms, are preferred.

Those triazolopyrimidines of the formula (I), in which
R¹ represents a residue of the formula 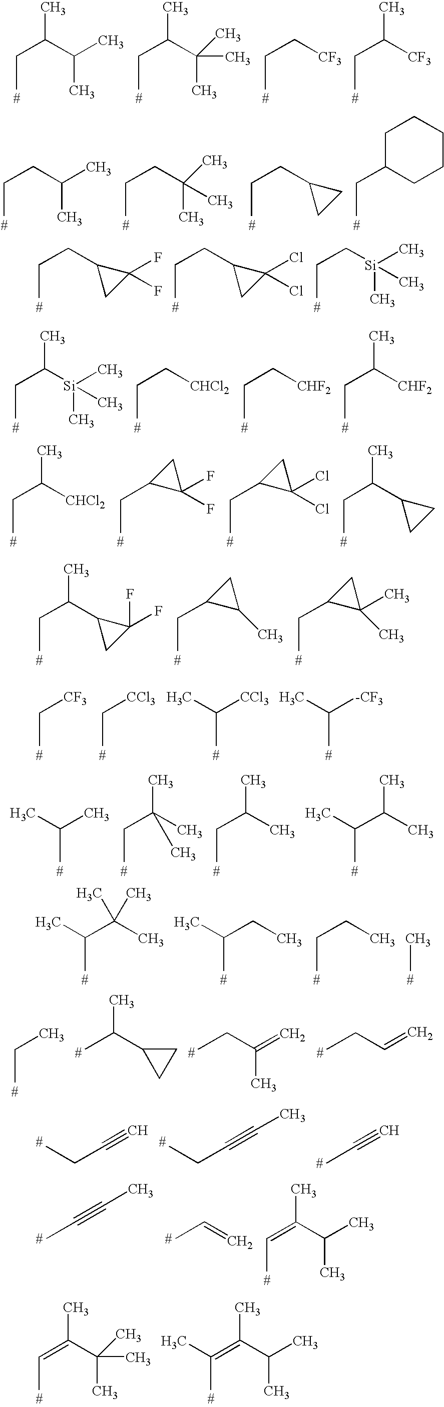 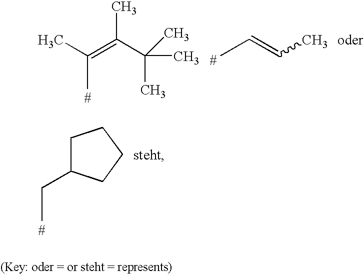
   or
R¹ represents a residue of the formula 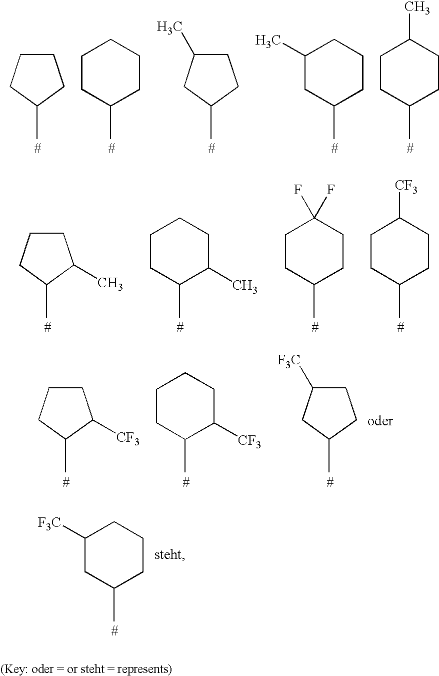
   or
R¹ represents a residue of the formula 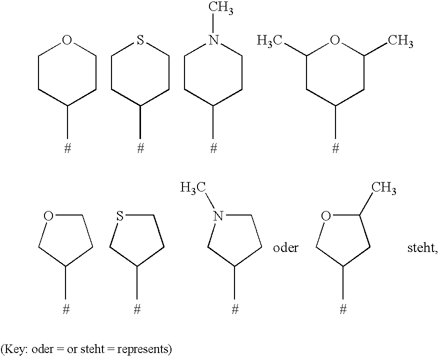
   # marking the linkage point in each case,
R² represents hydrogen, fluorine, chlorine, bromine, iodide, methyl, ethyl, isopropyl, cyclopropyl, cyclobutyl, cyclopentyl, cyclohexyl, trifluoromethyl, 1-trifluoromethyl-2,2,2-trifluorethyl, or heptafluoroisopropyl,
R³ represents pyridyl, which is linked in the second or fourth position and may be substituted one to four times, identically or differently, by fluorine, chlorine, bromine, cyano, nitro, methyl, ethyl, methoxy, methylthio, hydroximinomethyl, hydroximinoethyl, methoximinomethyl, methoximinoethyl and/or trifluoromethyl, or
R³ represents pyrimidyl, which is linked in the second or fourth position and may be substituted one to three times, identically or differently, by fluorine, chlorine, bromine, cyano, nitro, methyl, ethyl, methoxy, methylthio, hydroximinomethyl, hydroximinoethyl, methoximinomethyl, methoximinoethyl and/or trifluoromethyl, or
R³ represents thienyl, which is linked in the second or third position and may be substituted one to three times, identically or differently, by fluorine, chlorine, bromine, cyano, nitro, methyl, ethyl, methoxy, methylthio, hydroximinomethyl, hydroximinoethyl, methoximinomethyl, methoximinoethyl and/or trifluoromethyl, or
R³ represents thiazolyl, which is linked in the second, fourth, or fifth position and may be substituted once or twice, identically or differently, by fluorine, chlorine, bromine, cyano, nitro, methyl, ethyl, methoxy, methylthio, hydroximinomethyl, hydroximinoethyl, methoximinomethyl, methoximinoethyl and/or trifluoromethyl,
   and
X represents fluorine, chlorine, bromine, cyano, methyl, methoxy, or methylthio.

The above-mentioned residue definitions may be combined arbitrarily with one another. In addition, individual definitions may be dispensed with.

If one uses 5,7-dichloro-6-(5-chloropyrimidin-4-yl)-2-methyl-[1,2,4]triazolo[1,5-a]pyrimidine and 4-methylcyclohexyl magnesium bromide as starting materials, the course of the method (a) according to the present invention may be illustrated by the following formula scheme.

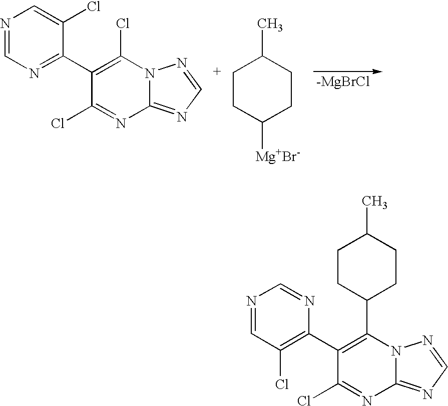

If one uses the above-mentioned compound produced according to the first step of the method (a) according to the present invention as the starting substance and sodium methylate as a reaction component, the course of the second step of the method (a) according to the present invention according to variation a may be illustrated by the following formula scheme.

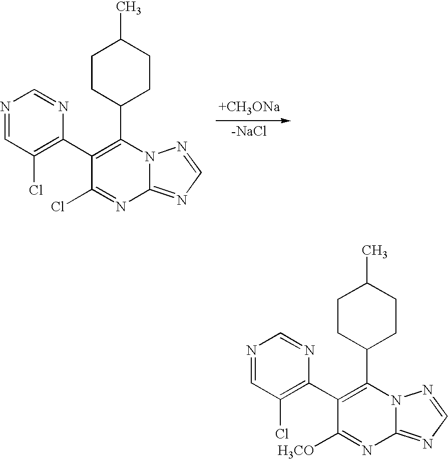

If one uses the above-mentioned compound produced according to the first step of the method (a) according to the present invention as the starting substance and methyl magnesium bromide as a reaction component, the course of the second step of the method (a) according to the present invention according to variation β may be illustrated by the following formula scheme.

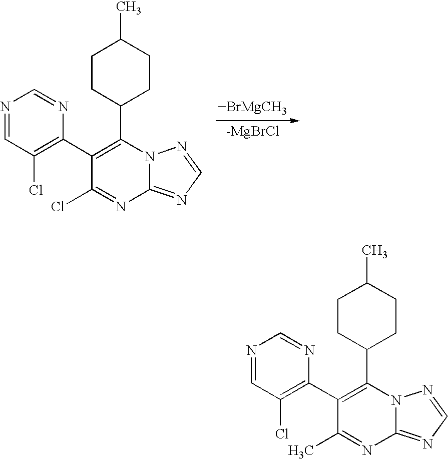

The dihalogen triazolopyrimidines necessary as starting materials when performing the method (a) according to the present invention are generally defined by the formula (II). In this formula (II), R² and R³ preferably have the meanings which were already cited as preferred for these residues in connection with the description of the materials according to the present invention of the formula (I). X¹ preferably represents fluorine, chlorine or bromine.

Y¹ preferably represents fluorine, chlorine or bromine, especially preferably fluorine or chlorine

The dihalogen triazolopyrimidines of the formula (II) are new. These materials are also suitable for combating undesired micro-organisms.

The dihalogen triazolopyrimidines of the formula (II) may be manufactured by reacting

(b) dihydroxy triazolopyrimidines of the formula 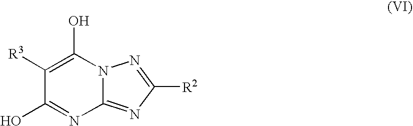
in which
R² and R³ have the meanings specified above,
with halogenation agents, optionally in the presence of a diluent.

If one uses 6-(5-chloropyrimidin-4-yl)-2-methyl[1,2,4]triazolo[1,5-a]-pyrimidin-5,7-diol as a starting material and phosphorus oxychloride mixed with phosphorus pentachloride as the halogenation agent, the course of the method (b) according to the present invention may be illustrated by the following formula scheme.

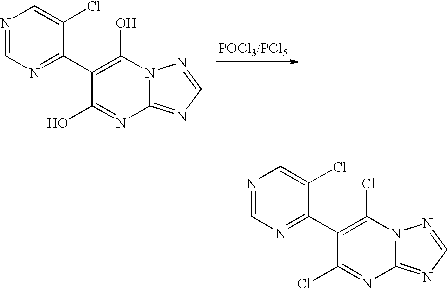

The dihydroxy triazolopyrimidines necessary as starting materials when performing the method (b) according to the present invention are generally defined by the formula (VI). In this formula, R² and R³ preferably have the meanings which were already cited as preferred for these residues in connection with the description of the materials according to the present invention of the formula (I).

The dihydroxy triazolopyrimidines of the formula (VI) are also previously unknown. They may be produced by reacting
(c) heterocyclyl malonic esters of the formula 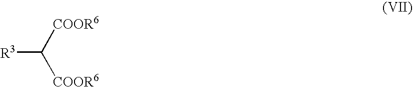
in which
R³ has the meaning specified above and
R⁶ represents alkyl having 1 to 4 carbon atoms,
   with aminotriazoles of the formula 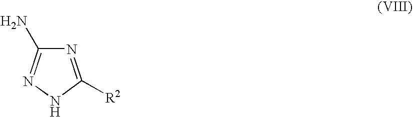
   in which
R² has the meaning specified above,
   optionally in the presence of a diluent and optionally in the presence of an acid binding agent.

If one uses 2-(5-chloropyrimidin-4-yl)-malonic dimethylester and 3-aminotriazole as the starting materials, the course of the method (c) according to the present invention may be illustrated by the following formula scheme.

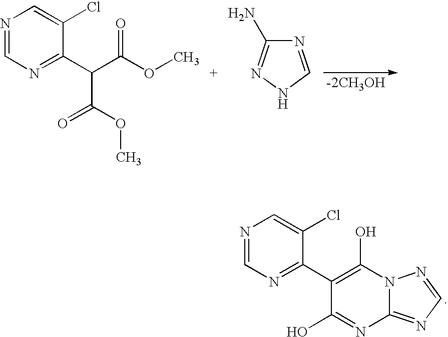

The heterocyclyl malonic esters necessary as starting materials for performing the method (c) according to the present invention are generally defined by the formula (VII). In this formula, R³ preferably has those meanings which were already cited as preferred for this residue in connection with the description of the materials according to the present invention of the formula (I). R⁶ preferably represents methyl or ethyl.

The heterocyclyl malonic esters of the formula (VII) are partially known (cf. DE-A 38 20 538-A, WO 01-11 965 and WO 99-32 464).

Pyridyl malonic esters of the formula 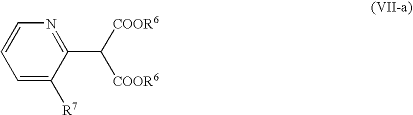
in which
R⁶ has the meaning specified above and
R⁷ represents halogen or halogenalkyl, are new.

Pyrimidyl malonic esters of the formula 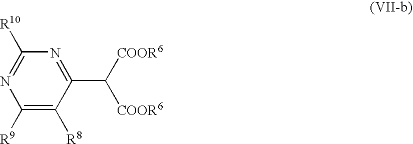
in which
R⁶ has the meaning specified above,
R⁸ represents halogen or halogenalkyl, and
R⁹ and R¹⁰ independently of one another, represent hydrogen, fluorine, chloride, bromine, methyl, ethyl or methoxy, are also new.

The pyridyl malonic esters of the formula (VII-a) may be produced by reacting
(d) halopyridines of the formula 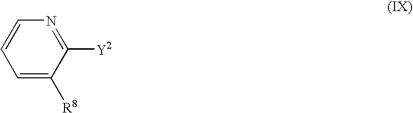
in which
R⁷ has the meaning specified above and
Y² represents halogen,
   with malonic esters of the formula 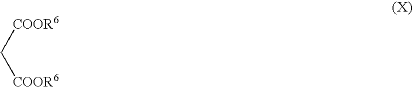
   in which
R⁶ has the meaning specified above,
   optionally in the presence of a diluent, optionally in the presence of a copper salt, and optionally in the presence of an acid acceptor.

If one uses 2-chloro-3-trifluoromethylpyridine and malonic acid dimethylester as the starting materials, the course of the method (d) according to the present invention may be illustrated by the following formula scheme.

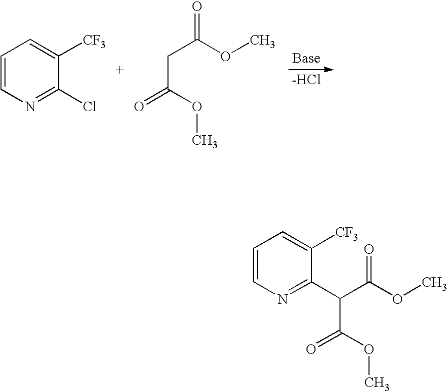

The halopyridines necessary as starting materials for performing the method (d) according to the present invention are generally defined by the formula (IX). In this formula, R⁷ preferably represents fluorine, chloride or trifluoromethyl. Y² preferably represents chloride or bromine.

The halopyridines of the formula (IX) are known synthetic chemicals.

The malonic acid esters of the formula (X), also necessary as starting materials for performing the method (d) according to the present invention, are also known synthetic chemicals.

The pyrimidyl malonic esters of the formula (VII-b) may be produced by reacting
(e) halopyrimidines of the formula 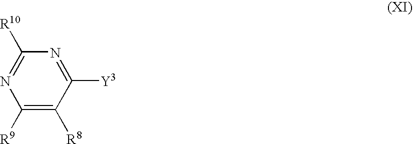
   in which
R⁸, R⁹ and R¹⁰ have the meanings specified above and
Y³ represents halogen,
   with malonic esters of the formula 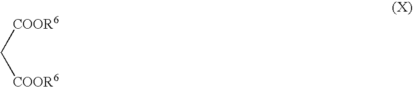
   in which
R⁶ has the meaning specified above,
   optionally in the presence of a diluent, optionally in the presence of a copper salt, and optionally in the presence of an acid acceptor.

If one uses 4,5-dichloropyrimidine and malonic dimethylester as the starting materials, the course of the method (e) according to the present invention may be illustrated by the following formula scheme.

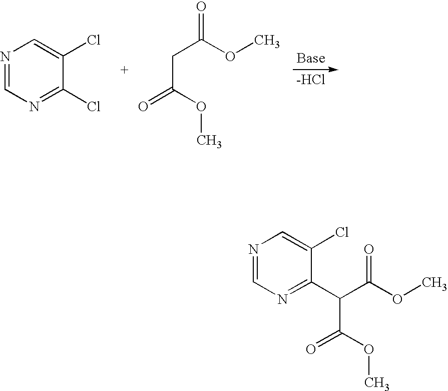

The halopyrimidines necessary as starting materials for performing the method (e) according to the present invention are generally defined by the formula (XI). In this formula, R⁸ preferably represents fluorine, chlorine or trifluoromethyl. R⁹ and R¹⁰ also, independently of one another, preferably represent hydrogen, fluorine, chlorine, bromine, methyl, ethyl or methoxy. Y³ preferably represents chlorine or bromine.

The halopyrimidines of the formula (XI) are known and may be produced according to known methods (cf. J. Chem. Soc. 1955, 3478-3481).

The aminotriazoles necessary as reaction components for performing the method (c) according to the present invention are generally defined by the formula (VII). In this formula, R² preferably has those meanings which were already cited as preferred for this residue in connection with the description of the materials of the formula (I) according to the present invention.

The aminotriazoles of the formula (VIII) are known or may be produced according to known methods (cf. DE-A 10 121 162).

All components typical for replacing hydroxy groups with halogen come into consideration as the halogenation agents when performing the method (b) according to the present invention. Phosphorus trichloride, phosphorus tribromide, phosphorus pentachloride, phosphorus oxychloride, thionyl chloride, thionyl bromide or their mixtures are preferably usable. The corresponding fluorine compounds of the formula (II) may be produced from the chlorine or bromine compounds through reaction with potassium fluoride.

The halogenation agents cited are known.

The compounds also necessary as reaction components for performing the method (a) according to the present invention are generally defined by the formula (III). In this formula, R¹ preferably has those meanings which were already specified as preferred for this residue in connection with the description of the compounds of the formula (I) according to the present invention. Me preferably also represents lithium, dihydroxyboranyl, a residue of the formula 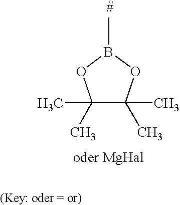
in which
Hal represents chlorine or bromine,

The metal compounds of the formula (III) are known or may be produced according to known methods.

The triazolopyrimidines necessary as starting materials when performing the second step of the method (a) according to the present invention are generally defined by the formula (Ia). In this formula, R¹, R² and R³ preferably have those meanings which were already cited in connection with the description of the materials according to the present invention of the formula (I). X¹ preferably represents fluorine, chlorine or bromine.

The compounds necessary as reaction components when performing the second step of the method according to the present invention (a, variation α) are generally defined by the formula (IV). In this formula, R⁴ preferably represents cyano, alkoxy having 1 to 4 carbon atoms, alkylthio having 1 to 4 carbon atoms, alkylsulphinyl having 1 to 4 carbon atoms, or alkylsulphonyl having 1 to 4 carbon atoms. Me¹ preferably also represents sodium or potassium.

In the formula (IV), R⁴ especially preferably represents cyano, methoxy or methylthio. Me¹ also especially preferably represents sodium or potassium.

The compounds of the formula (IV) are known.

The Grignard compounds necessary as reaction components when performing the second step of the method (a, variation β) according to the present invention are generally defined by the formula (V). In this formula, R⁵ preferably represents alkyl having 1 to 4 carbon atoms, especially preferably methyl. Hal¹ preferably and especially preferably represents chlorine or bromine.

The Grignard compounds of the formula (V) are known or may be produced according to known methods, expediently directly before their use for further synthesis

All typical inert organic solvents come into consideration as diluents when performing the method (a) according to the present invention. Ethers are preferably usable, such as diethylether, diisopropylether, methyl-t-butylether, methyl-t-amylether, dioxane, tetrahydrofuran, 1,2-dimethoxyethane, 1,2-diethoxyethane or anisol; amides, such as N,N-dimethylformamide, N,N-dimethylacetamide, N-methylformanilide, N-methylpyrrolidone or hexamethyl phosphoric triamide.

All inorganic or organic bases typical for reactions in this type come into consideration as acid acceptors when performing the method (a) according to the present invention. Alkaline earth metal or alkali metal hydroxides, acetates, carbonates, hydrogen carbonates or phosphates, such as sodium hydroxide, potassium hydroxide, sodium acetate, sodium carbonate, potassium carbonate, potassium hydrogen carbonate, sodium hydrogen carbonate, cesium carbonate, or silver phosphate are preferably usable.

All typical reaction accelerators for reactions of this type come into consideration as catalysts when performing the first step of the method (a) according to the present invention. Palladium, nickel, copper, or iron salts and/or complexes are preferably usable. Examples of these are copper(I) chloride, copper(I) bromide, copper(I) iodide, copper(I) cyanide, iron(III) acetate, tetrakis-(triphenylphosphine) palladium, bis(triphenylphosphine) palladium dichloride and 1,1′-bis(diphenylphosphino)ferrocene palladium(II) chloride.

Palladium or nickel complexes which are produced in the reaction mixture by adding a palladium or a nickel salt and a substance which functions as a complexing ligand separately to the reaction mixture are also preferably usable. Examples of ligand producers are:

Triethylphosphane, tri-tert.-butylphosphane, tricyclohexylphosphane, 2-(dicyclohexylphosphane) biphenyl, 2-(di-tert.-butylphosphane) biphenyl, 2-(dicyclohexylphosphane)-2′-(N,N-dimethylamino)-biphenyl, triphenylphosphane, tris-(o-tolyl)-phosphane, sodium 3-(diphenylphosphino)benzolsulphonate, tris-2-(methoxyphenyl)-phosphane, 2,2′-bis-(diphenylphosphane)-1,1′-binaphthyl, 1,4-bis-(diphenylphosphane)-butane, 1,2-bis-(diphenylphosphane)-ethane, 1,4-bis-(dicyclohexylphosphane)-butane, 1,2-bis-(dicyclohexylphosphane)-ethane, 2-(dicyclohexylphosphane)-2′-(N,N-dimethylamino)-biphenyl, bis(diphenylphosphino)ferrocene and tris-(2,4-tert.-butylphenyl)-phsophite.

The reaction temperatures may be varied in a wide range when performing the method (a) according to the present invention. In general, one operates at temperatures between 0° C. and 150° C., preferably at temperatures between 0° C. and 80° C.

When performing the method (a) according to the present invention, generally 1 to 10 mol, preferably 1 to 3 mol of a metal compound of the formula (III) is used for 1 mol of dihalogen triazolopyrimidine of the formula (II). The workup is performed according to typical methods.

All solvents typical for halogenations of this type come into consideration as diluents when performing the second step of the method (a, variation α) according to the present invention. Halogenated hydrocarbons are preferably usable, such as chlorobenzene, dichlorobenzene, dichloromethane, chloroform, tetrachloromethane, dichloroethane or trichloroethane; ethers, such as diethylether, diisopropylether, methyl-t-butylether, methyl-t-amylether, dioxane, tetrahydrofuran, 1,2-dimethoxyethane, 1,2-diethoxyethane or anisol; nitriles, such as acetonitrile, propionitrile, n- or i-butyronitrile or benzonitrile; amides, such as N,N-dimethylformamide, N,N-dimethylacetamide, N-methylformanilide, N-methylpyrrolidone or hexamethyl phosphoric triamide; esters such as acetic methyl ester or acetic ethyl ester; sulphoxides, such as dimethyl sulphoxide; sulphones, such as sulpholan.

The temperatures may also be varied in a wide range when performing the second step of the method (a, variation α) according to the present invention. In general, one operates at temperatures between 0° C. and 150° C., preferably between 20° C. and 100° C.

When performing the second step of the method (a, variation α) according to the present invention, triazolopyrimidine of the formula (Ia) is generally reacted with an excess of a compound of the formula (IV). The workup is performed according to typical methods.

When performing the second step of the method (a, variation β) according to the present invention, all solvents typical for Grignard reactions come into consideration as the diluent. Ethers, such as diethyl ether, are preferably usable.

The reaction temperatures may be varied in a specific range when performing the second step of the method (a, variation β) according to the present invention. In general, one operates at temperatures between −20° C. and 80° C., preferably between 0° C. and 60° C.

When performing the second step of the method (a, variation β) according to the present invention, triazolopyrimidine of the formula (Ia) is reacted with an equivalent quantity or with an excess of a Grignard compound of the formula (V). The workup is again performed according to typical methods.

All solvents typical for halogenations of this type come into consideration as diluents when performing the method (b) according to the present invention. Halogenated aliphatic or aromatic hydrocarbons, such as chlorobenzene, are preferably usable. However, the halogenation agent itself, e.g., phosphorus oxychloride, or a mixture of the halogenation agents may function as the diluent.

The temperatures may also be varied in a wide range when performing the method (b) according to the present invention. In general, one operates at temperatures between 0° C. and 150° C., preferably between 10° C. and 120° C.

When performing the method (b) according to the present invention, dihydroxy triazolopyrimidine of the formula (VI) is generally reacted with an excess of halogenation agent. The workup is performed according to typical methods.

All inert organic solvents typical for reactions of this type come into consideration as diluents when performing the method (c) according to the present invention. Alcohols, such as methanol, ethanol, n-propanol, i-propanol, n-butanol and tert.-butanol, are preferably usable.

All inorganic and organic bases typical for reactions of this type come into consideration as acid binders when performing the method (c) according to the present invention. Tertiary amines, such as tributylamine or pyridine, are preferably usable. Amine used in excess may also function as a diluent.

The temperatures may be varied in a wide range when performing the method (b) according to the present invention. In general, one operates at temperatures between 20° C. and 200° C., preferably between 50° C. and 180° C.

When performing the method (c) according to the present invention, heterocyclyl malonic ester of the formula (VII) and aminotriazole of the formula (VIII) are generally reacted in equivalent quantities. However, it is also possible to use one or the other component in excess. The workup is performed according to typical methods.

All typical inert organic solvents come into consideration as the diluent when performing the methods (d) and (e) according to the present invention. Halogenated hydrocarbons, such as chlorobenzene, dichlorobenzene, dichloromethane, chloroform, tetrachloromethane, dichloroethane or trichlorethane; ethers, such as diethylether, diisopropylether, methyl-t-butylether, methyl-t-amylether, dioxane, tetrahydrofuran, 1,2-dimethoxyethane, 1,2-diethoxyethane or anisole; nitriles, such as acetonitrile, propionitrile, n- or i-butyronitrile or benzonitrile; amides, such as N,N-dimethylformamide, N,N-dimethylacetamide, N-methylformianilid, N-methylpyrrolidone or hexamethyl phosphoric triamide; sulphoxides, such as dimethylsulphoxide; sulphones, such as sulpholane; alcohols, such as methanol, ethanol, n- or i-propanol, n-, i-, sec- or tert-butanol, ethanediol, propane-1,2-diol, ethoxyethanol, methoxyethanol, diethylene glycol monomethylether, diethylene glycol monoethylether, their mixtures with water or even pure water are preferably usable.

The particular typical copper salts come into consideration as copper salts when performing the methods (d) and (e) according to the present invention. Copper(I) chloride or copper(I) bromide are preferably usable.

All inorganic or organic bases typical for reactions in this type come into consideration as acid acceptors when performing the methods (d) and (e) according to the present invention. Alkaline earth metal or alkali metal hydrides, hydroxides, amides, alcoholates, acetates, carbonates or hydrogen carbonates, such as sodium hydride, sodium amide, lithium diisopropylamide, sodium methylate, sodium ethylate, potassium tert.-butylate, sodium hydroxide, potassium hydroxide, sodium acetate, potassium acetate, calcium acetate, sodium carbonate, potassium carbonate, potassium hydrogen carbonate and sodium hydrogen carbonate, and additionally ammonium compounds such as ammonium hydroxide, ammonium acetate and ammonium carbonate, as well as tertiary amines, such as trimethylamine, triethylamine, tributylamine, N,N-dimethylaniline, N,N-dimethyl-benzylamine, pyridine, N-methylpiperidine, N-methylmorpholine, N,N-dimethylaminopyridine, diazabicyclooctane (DABCO), diazabicyclononene (DBN) or diazabicycloundecene (DBU) are preferably usable.

The reaction temperatures may be varied in a wide range when performing the methods (d) and (e) according to the present invention. In general, one operates at temperatures between 0° C. and 150° C., preferably at temperatures between 0° C. and 80° C.

When performing the method (d) according to the present invention, generally 1 to 15 mol, preferably 1.3 to 8 mol of malonic ester of the formula (IX) is used for 1 mol of malonic ester of the formula (X). The workup is performed according to typical methods.

When performing the method (e) according to the present invention, generally 1 to 15 mol, preferably 1.3 to 8 mol of malonic ester of the formula (X) is used for 1 mol of halopyrimidine of the formula (XI). The workup is performed according to typical methods.

The methods according to the present invention are generally performed at atmospheric pressure. However, it is also possible to work at elevated pressure.

The materials according to the present invention have a strong microbicidal effect and may be used for combating undesired micro-organisms, such as fungi and bacteria, in plant protection, and in material protection.

Fungicides may be used in plant protection for combating Plasmodiophoromycetes, Oomycetes, Chytridiomycetes, Zygomycetes, Ascomycetes, Basidiomycetes and Deuteromycetes.

Bactericides may be used in plant protection for combating Pseudomonadaceae, Rhizobiaceae, Enterobacteriaceae, Corynebacteriaceae and Streptomycetaceae.

Some pathogens of fungal and bacterial diseases, which fall under the generic terms listed above, will be listed as examples, but not as restrictions:

Xanthomonas species, such as Xanthomonas campestris pv. oryzae;

Pseudomonas species, such as Pseudomonas syringae pv. lachrymans;

Erwinia species, such as Erwinia amylovora;

Pythium species, such as Pythium ultimum;

Phytophthora species, such as Phytophthora infestans;

Pseudoperonospora species, such as Pseudoperonospora humuli or

Pseudoperonospora cubensis;

Plasmopara species, such as Plasmopara viticola;

Bremia species, such as Bremia lactucae;

Peronospora species, such as Peronospora pisi or P. brassicae;

Erysiphe species, such as Erysiphe graminis;

Sphaerotheca species, such as Sphaerotheca fuliginea;

Podosphaera species, such as Podosphaera leucotricha;

Venturia species, such as Venturia inaequalis;

Pyrenophora species, such as Pyrenophora teres or P. graminea

(conidia form: Drechslera, syn: Helminthosporium);

Cochliobolus species, such as Cochliobolus sativus

(conidia form: Drechslera, syn: Helminthosporium);

Uromyces species, such as Uromyces appendiculatus;

Puccinia species, such as Puccinia recondita;

Sclerotinia species, such as Sclerotinia sclerotiorum;

Tilletia species, such as Tilletia caries;

Ustilago species, such as Ustilago nuda or Ustilago avenae;

Pellicularia species, such as Pellicularia sasakii;

Pyricularia species, such as Pyricularia oryzae;

Fusarium species, such as Fusarium culmorum;

Botrytis species, such as Botrytis cinerea;

Septoria species, such as Septoria nodorum;

Leptosphaeria species, such as Leptosphaeria nodorum;

Cercospora species, such as Cercospora canescens;

Alternaria species, such as Alternaria brassicae;

Pseudocercosporella species, such as Pseudocercosporella herpotrichoides.

The active ingredients according to the present invention also have a very good strengthening effect in plants. They are therefore suitable for mobilizing plant defenses against infection by undesired micro-organisms.

Plant-strengthening (resistance-inducing) materials are to be understood in the present context as those substances which are capable of stimulating the defense system of plants in such a way that, upon subsequent inoculation with undesired micro-organisms, the treated plants unfold extensive resistance to these micro-organisms.

In the present case, undesired micro-organisms are to be understood as phytopathogenic fungi, bacteria, and viruses. The materials according to the present invention may thus be used for protecting plants against infection by the pathogens cited within a certain period of time after treatment. The period of time within which this protection is provided generally extends from 1 to 10 days, preferably 1 to 7 days after the treatment of the plants with the active ingredients.

The good phytotolerance of the active ingredients in the concentrations necessary for combating plant diseases allows treatment of aboveground plant parts, of plants and seeds, and of the soil.

In this case, the active ingredients according to the present invention may be used especially successfully for combating grain diseases, such as Erysiphe species, and of diseases in wine, fruit, and vegetable farming, such as Botrytis, Venturia, Sphaerotheca and Podosphaera species.

The active ingredients according to the present invention are also suitable for increasing the harvest yield. They also have low toxicity and good phytotolerance.

The active ingredients according to the present invention may optionally also be used in specific concentrations and applied quantities as herbicides, to influence plant growth, and to combat animal pests. They may also be used as intermediate and precursor products for synthesizing further active ingredients if necessary.

According to the present invention, all plants and plant parts may be treated. Plants are understood in this case as all plants and plant populations, such as desired and undesired wild plants or cultured plants (including naturally occurring cultured plants). Cultured plants may be plants which are obtained through conventional cultivation and optimization methods or through methods of biotechnology and genetic engineering or combinations of these methods, including transgenic plants and including plant species which may or may not be protected by species protection rights. Plant parts are to be understood as all aboveground and below ground parts and organs of the plants, such as sprouts, leaves, flowers, and roots, for example, leaves, needles, stakes, stems, flowers, fruits, and seeds, as well as roots, bulbs, and rhizomes being listed. The plant parts also include hereditary material as well as vegetative and generative propagation material, such as slips, bulbs, rhizomes, cuttings, and seeds.

The treatment of the plants and plant parts according to the present invention using the active ingredients is performed directly or through the effect on their environment, living space, or storage space according to the typical treatment methods, e.g., through dipping, spraying, vaporizing, misting, scattering, painting, and for propagation material, particularly for seeds, also through single-layer or multilayered enveloping.

In material protection, the materials according to the present invention may be used for protecting technical materials against infection and destruction by undesired micro-organisms.

Technical materials are to be understood in the present context as inanimate materials which have been prepared for use in technology. For example, technical materials which may be protected by active ingredients according to the present invention from microbial change or destruction are adhesives, glues, paper and cardboard, textiles, leather, wood, paints and plastic articles, coolants, and other materials which may be infected or destroyed by micro-organisms. Parts of production facilities, such as coolant water loops, which may be impaired by reproduction of micro-organisms, are also cited in the scope of the materials to be protected. Preferably, adhesives, glues, paper and cardboard, leather, wood, paints, coolants, and thermal transfer fluids are cited as technical materials in the scope of the present invention, especially preferably wood.

For example, bacteria, fungi, yeasts, algae, and slime organisms are cited as micro-organisms which may cause degradation or change of the technical materials. Preferably, the active ingredients according to the present invention act against fungi, particularly mold fungi, wood-staining and wood-destroying fungi (Basidiomycetes), and against slime organisms and algae.

Micro-organisms of the following species are cited as examples:

Alternaria, such as Alternaria tenuis,

Aspergillus, such as Aspergillus niger,

Chaetomium, such as Chaetomium globosum,

Coniophora, such as Coniophora puetana,

Lentinus, such as Lentinus tigrinus,

Penicillium, such as Penicillium glaucum,

Polyporus, such as Polyporus versicolor,

Aureobasidium, such as Aureobasidium pullulans,

Sclerophoma, such as Sclerophoma pityophila,

Trichoderma, such as Trichoderma viride,

Escherichia, such as Escherichia coli,

Pseudomonas, such as Pseudomonas aeruginosa,

Staphylococcus, such as Staphylococcus aureus.

As a function of their particular physical and/or chemical properties, the active ingredients may be converted into the typical formulations, such as solvents, emulsions, suspensions, powders, foams, pastes, granules, aerosols, extremely fine encapsulations in polymer materials, and into envelope compounds for seeds, as well as ULV cold and hot mist formulations.

These formulations are produced in ways known per se, e.g., by mixing the active ingredients with extenders, i.e., liquid solvents, liquefied gases under pressure, and/or solid carrier materials, optionally using surfactants, i.e., and also emulsifiers and/or dispersing agents and/or foam-producing agents. If water is used as an extender, organic solvents may also be used as an auxiliary solvents, for example. The following solvents essentially come into consideration as the liquid solvent: aromatics, such as xylene, toluene or alkylnaphthaline, chlorinated aromatics or chlorinated aliphatic hydrocarbons, such as chlorobenzene, chloroethylene or methylene chloride, aliphatic hydrocarbons, such as cyclohexane, or paraffins, such as petroleum fractions, alcohols, such as butanol or glycol as well as their ethers and esters, ketones, such as acetone, methylethylketone, methylisobutylketone or cyclohexanone, strongly polar solvents, such as dimethylformamide and dimethylsulphoxide, as well as water. Liquefied gaseous extenders or carriers are those liquids which are gaseous at normal temperature and under normal pressure, such as aerosol propellant gases, such as halogenated hydrocarbons as well as butane, propane, nitrogen and carbon dioxide. The following materials come into consideration as solid carriers: for example, natural rock flours, such as kaolin, aluminum oxide, talcum, chalk, quartz, attapulgite, montmorillonite or diatomaceous earths and synthetic rock flours, such as highly dispersed silicic acid, aluminum oxide and silicates. The following materials come into consideration as solid carriers for granules: for example, broken and fractionated natural stones such as calcite, pumice, marble, sepiolite, dolomite, as well as synthetic granulates made of inorganic and organic flours and granulates made of organic material like sawdust, coconut shells, maize cobs, and tobacco stalks. The following materials come into consideration as emulsifiers and/or foam-producing agents: for example, non-ionogenic and anionic emulsifiers, such as polyoxyethylene fatty acid esters, polyoxyethylene fatty alcohol ethers, e.g., alkylaryl polyglycolethers, alkyl sulphonates, alkyl sulphates, aryl sulphonates and protein hydrolysates. The following materials come into consideration as dispersing agents: e.g., lignin sulphite waste liquors and methyl cellulose.

Adhesives such as carboxymethylcellulose, natural and synthetic powdered, grainy, or latex polymers may be used in the formulations, such as gum arabic, polyvinylalcohol, polyvinylacetate, as well as natural phospholipids, such as kephalins and lecithins, and synthetic phospholipids. Further additives may be mineral and vegetable oils.

Coloring agents such as inorganic pigments, e.g., iron oxide, titanium oxide, ferrocyanide blue, and organic coloring agents such as alizarin, azo and metal phthalocyanine coloring agents and trace nutrients, such as salts of iron, manganese, boron, copper, cobalt, molybdenum, and zinc may be used.

The formulations generally contain between 0.1 and 95 percent by weight active ingredient, preferably between 0.5 and 90%.

The active ingredients according to the present invention may also be used per se or in their formulations with known fungicides, bactericides, acaricides, nematicides or insecticides, in order to thus broaden the activity spectrum or avoid the development of resistance, for example. In many cases, synergistic effects are achieved in this case, i.e., the effectiveness of the mixture is greater than the effectiveness of the individual components.

The following compounds come into consideration as mixing partners, for example:

Fungicides:

2-phenylphenol; 8-hydroxychinolinsulphat;

acibenzenear-5-methyl; aldimorph; amidoflumet; ampropylfos; ampropylfos-potassium; andoprim; anilazine; azaconazole; azoxystrobin;

benalaxyl; benodanil; benomyl; benthiavalicarb isopropyl; benzamacril; benzamacril-isobutyl; bilanafos; binapacryl; biphenyl; bitertanol; blasticidin-s; bromuconazole; bupirimate; buthiobate; butylamine;

calcium polysulphide; capsimycin; captafol; captan; carbendazim; carboxin; carpropamid; carvone; chinomethionat; chlobenthiazone; chlorofenazole; chloroneb; chlorothalonil; chlozolinate; clozylacon; cyazofamid; cyflufenamid; cymoxanil; cyproconazole; cyprodinil; cyprofuram;

Dagger G; debacarb; dichlofluanid; dichione; dichlorophen; diclocymet; diclomezine; dicloran; diethofencarb; difenoconazole; diflumetorim; dimethirimol; dimethomorph; dimoxystrobin; diniconazole; diniconazole-m; dinocap; diphenylamine; dipyrithione; ditalimfos; dithianon; dodine; drazoxolon;

edifenphos; epoxiconazole; ethaboxam; ethirimol; etridiazole;

famoxadone; fenamidone; fenapanil; fenarimol; fenbuconazole; fenfuram; fenhexamid; fenitropan; fenoxanil; fenpiclonil; fenpropidin; fenpropimorph; ferbam; fluazinam; flubenzimine; fludioxonil; flumetover; flumorph; fluoromide; fluoxastrobin; fluquinconazole; flurprimidol; flusilazole; flusulphamide; flutolanil; flutriafol; folpet; fosetyl-A1; fosetyl sodium; fuberidazole; furalaxyl; furametpyr; furcarbanil; furmecyclox;

guazatine;

hexachlorobenzene; hexaconazole; hymexazol;

imazalil; imibenconazole; iminoctadine triacetate; iminoctadine tris(albesil); iodocarb; ipconazole; iprobenfos; iprodione; iprovalicarb; irumamycin; isoprothiolane; isovaledione;

kasugamycin; kresoxim-methyl;

mancozeb; maneb; meferimzone; mepanipyrim; mepronil; metalaxyl; metalaxyl-m; metconazole; methasulphocarb; methfuroxam; metiram; metominostrobin; metsulphovax; mildiomycin; myclobutanil; myclozolin;

natamycin; nicobifen; nitrothal-isopropyl; noviflumuron; nuarimol;

ofurace; orysastrobin; oxadixyl; oxolinic acid; oxpoconazole; oxycarboxin; oxyfenthiin;

paclobutrazol; pefurazoate; penconazole; pencycuron; phosdiphen; phthalide; picoxystrobin; piperalin; polyoxins; polyoxorim; probenazole; prochloraz; procymidone; propamocarb; propanosine-sodium; propiconazole; propineb; proquinazid; prothioconazole; pyraclostrobin; pyrazophos; pyrifenox; pyrimethanil; pyroquilon; pyroxyfur; pyrrolnitrine;

quinconazole; quinoxyfen; quintozene;

simeconazole; spiroxamine; sulphur;

tebuconazole; tecloftalam; tecnazene; tetcyclacis; tetraconazole; thiabendazole; thicyofen; thifluzamide; thiophanate-methyl; thiram; tioxymid; tolclofos-methyl; tolylfluanid; triadimefon; triadimenol; triazbutil; triazoxide; tricyclamide; tricyclazole; tridemorph; trifloxystrobin; triflumizole; triforine; triticonazole;

uniconazole;

validamycin a; vinclozolin;

zineb; ziram; zoxamide;

(2S)—N-[2-[4-[[3-(4-chlorophenyl)-2-propinyl]oxy]-3-methoxyphenyl]ethyl]-3-methyl-2-[(methylsulphonyl)amino]-butanamide;
1-(1-naphthalenyl)-1H-pyrrol-2,5-dion;
2,3,5,6-tetrachlor-4-(methylsulphonyl)-pyridine;
2-amino-4-methyl-n-phenyl-5-thiazolcarboxamide;
2-chloro-n-(2,3-dihydro-1,1,3-trimethyl-1H-inden-4-yl)-3-pyridincarboxamide;
3,4,5-trichloro-2,6-pyridindicarbonitrile;
   actinovate;
cis-1-(4-chlorophenyl)-2-(1H-1,2,4-triazol-1-yl)-cycloheptanol;
methyl 1-(2,3-dihydro-2,2-dimethyl-1H-inden-1-yl)-1H-imidazol-5-carboxylate;
   monopotassium carbonate;
n-(6-methoxy-3-pyridinyl)-cyclopropancarboxamide;
   sodium tetrathiocarbonate;
   as well as copper salts and preparations, such as Bordeaux mixture; copper hydroxide; copper naphthenate; copper oxychloride; copper sulphate; cufraneb; copper oxide; mancopper; oxine copper.
   Bactericides:
   bronopol, dichlorophen, nitrapyrin, nickel dimethyldithiocarbamate, kasugamycin, octhilinon, furan carboxylic acid, oxytetracyclin, probenazol, streptomycin, tecloftalam, copper sulphate and other copper preparations.
   Insecticides/Acaricides/Nematicides:
   abamectin, ABG-9008, acephate, acequinocyl, acetamiprid, acetoprole, acrinathrin, AKD-1022, AKD-3059, AKD-3088, alanycarb, aldicarb, aldoxycarb, allethrin, allethrin 1R-isomers, alpha-cypermethrin (alphamethrin), amidoflumet, aminocarb, amitraz, avermectin, AZ-60541, azadirachtin, azamethiphos, azinphos-methyl, azinphos-ethyl, azocyclotin,
   Bacillus popilliae, Bacillus sphaericus, Bacillus subtilis, Bacillus thuringiensis, Bacillus thuringiensis strain EG-2348, Bacillus thuringiensis strain GC-91, Bacillus thuringiensis strain NCTC-11821, baculoviruses, Beauveria bassiana, Beauveria tenella, bendiocarb, benfuracarb, bensultap, benzoximate, beta-cyfluthrin, beta-cypermethrin, bifenazate, bifenthrin, binapacryl, bioallethrin, bioallethrin-5-cyclopentyl-isomer, bioethanomethrin, biopermethrin, bioresmethrin, bistrifluron, BPMC, brofenprox, bromophos ethyl, bromopropylate, bromfenvinfos (methyl), BTG-504, BTG-505, bufencarb, buprofezin, butathiofos, butocarboxim, butoxycarboxim, butylpyridaben,
   cadusafos, camphechlor, carbaryl, carbofuran, carbophenothion, carbosulphan, cartap, CGA-50439, chinomethionat, chlordane, chlordimeform, chloethocarb, chlorethoxyfos, chlorfenapyr, chlorfenvinphos, chlorfluazuron, chlormephos, chlorobenzilate, chloropicrin, chlorproxyfen, chlorpyrifos methyl, chlorpyrifos (ethyl), chlovaporthrin, chromafenozide, cis-cypermethrin, cis-resmethrin, cis-permethrin, clocythrin, cloethocarb, clofentezine, clothianidin, clothiazoben, codlemone, coumaphos, cyanofenphos, cyanophos, cycloprene, cycloprothrin, cydia pomonella, cyfluthrin, cyhalothrin, cyhexatin, cypermethrin, cyphenothrin (1R-trans-isomer), cyromazine,
   DDT, deltamethrin, demeton-5-methyl, demeton-5-methylsulphon, diafenthiuron, dialifos, diazinon, dichlofenthion, dichlorvos, dicofol, dicrotophos, dicyclanil, diflubenzuron, dimethoate, dimethylvinphos, dinobuton, dinocap, dinotefuran, diofenolan, disulphoton, docusat-sodium, dofenapyn, DOWCO-439,
   eflusilanate, emamectin, emamectin-benzoate, empenthrin (1R-isomer), endosulphan, Entomopthora spp., EPN, esfenvalerate, ethiofencarb, ethiprole, ethion, ethoprophos, etofenprox, etoxazole, etrimfos,
   famphur, fenamiphos, fenazaquin, fenbutatin oxide, fenfluthrin, fenitrothion, fenobucarb, fenothiocarb, fenoxacrim, fenoxycarb, fenpropathrin, fenpyrad, fenpyrithrin, fenpyroximate, fensulphothion, fenthion, fentrifanil, fenvalerate, fipronil, flonicamid, fluacrypyrim, fluazuron, flubenzimine, flubrocythrinate, flucycloxuron, flucythrinate, flufenerim, flufenoxuron, flufenprox, flumethrin, flupyrazofos, flutenzin (flufenzine), fluvalinate, fonofos, formetanate, formothion, fosmethilan, fosthiazate, fubfenprox (fluproxyfen), furathiocarb,
   gamma HCH, gossyplure, grandlure, granulose viruses,
   halfenprox, halofenozide, HCH, HCN-801, heptenophos, hexaflumuron, hexythiazox, hydramethylnone, hydroprene,
   IKA-2002, imidacloprid, imiprothrin, indoxacarb, iodofenphos, iprobenfos, isazofos, isofenphos, isoprocarb, isoxathion, ivermectin,
   japonilure,
   kadethrin, nuclear polyhedrosis viruses, kinoprene,
   lambda cyhalothrin, lindane, lufenuron,
   malathion, mecarbam, mesulphenfos, metaldehyde, metam-sodium, methacrifos, methamidophos, metharhizium anisopliae, metharhizium flavoviride, methidathion, methiocarb, methomyl, methoprene, methoxychlor, methoxyfenozide, metolcarb, metoxadiazone, mevinphos, milbemectin, milbemycin, MKI-245, MON-45700, monocrotophos, moxidectin, MTI-800,
   naled, NC-104, NC-170, NC-184, NC-194, NC-196, niclosamide, nicotine, nitenpyram, nithiazine, NNI-0001, NNI-0101, NNI-0250, NNI-9768, novaluron, noviflumuron,
   OK-5101, OK-5201, OK-9601, OK-9602, OK-9701, OK-9802, omethoate, oxamyl, oxydemeton-methyl,
   Paecilomyces fumosoroseus, parathion methyl, parathion (ethyl), permethrin (cis-, trans-), petroleum, PH-6045, phenothrin (1R-trans isomer), phenthoate, phorate, phosalone, phosmet, phosphamidon, phosphocarb, phoxim, piperonyl butoxide, pirimicarb, pirimiphos methyl, pirimiphos ethyl, prallethrin, profenofos, promecarb, propaphos, propargite, propetamphos, propoxur, prothiofos, prothoate, protrifenbute, pymetrozine, pyraclofos, pyresmethrin, pyrethrum, pyridaben, pyridalyl, pyridaphenthion, pyridathion, pyrimidifen, pyriproxyfen,
   quinalphos,
   resmethrin, RH-5849, ribavirin, RU-12457, RU-15525,
   S-421, S-1833, salithion, sebufos, SI-0009, silafluofen, spinosad, spirodiclofen, spiromesifen, sulphluramid, sulphotep, sulprofos, SZI-121,
   tau-fluvalinate, tebufenozide, tebufenpyrad, tebupirimfos, teflubenzuron, tefluthrin, temephos, temivinphos, terbam, terbufos, tetrachlorvinphos, tetradifon, tetramethrin, tetramethrin (1R isomer), tetrasul, theta-cypermethrin, thiacloprid, thiamethoxam, thiapronil, thiatriphos, thiocyclam hydrogen oxalate, thiodicarb, thiofanox, thiometon, thiosultap sodium, thuringiensin, tolfenpyrad, tralocythrin, tralomethrin, transfluthrin, triarathene, triazamate, triazophos, triazuron, trichlophenidine, trichlorfon, triflumuron, trimethacarb,
   vamidothion, vaniliprole, verbutin, Verticillium lecanii,
   WL-108477, WL-40027,
   YI-5201, YI-5301, YI-5302,
   XMC, xylylcarb,
   ZA-3274, zeta-cypermethrin, zolaprofos, ZXI-8901,
   the compound 3-methyl-phenyl-propylcarbamate (Tsumacide Z),
   the compound 3-(5-chloro-3-pyridinyl)-8-(2,2,2-trifluorethyl)-8-azbicyclo[3.2.1]octane-3-carbonitrile (CAS-Reg.-No. 185982-80-3) and the corresponding 3-endo-isomers (CAS-Reg.-No. 185984-60-5) (cf. WO-96/37494, WO-98/25923),
   as well as preparations which contain insecticidally active plant extracts, nematodes, fungi, or viruses.

A mixture with other known active ingredients, such as herbicides, or with fertilizers and growth regulators, safeners, and/or semiochemicals is also possible.

In addition, the compounds of the formula (I) according to the present invention also have very good antimycotic effect. They have a very broad antimycotic activity spectrum, particularly against dermatophytes and sprout fungi, mold and diphasic fungi (e.g., against Candida species such as Candida albicans, Candida glabrata) as well as Epidermophyton floccosum, Aspergillus species such as Aspergillus niger and Aspergillus fumigatus, Trichophyton species such as Trichophyton mentagrophytes, Microsporon species such as Microsporon canis and audouinii. The list of these fungi does not represent a restriction of the mycotic spectrum which may be contained, but rather only has explanatory character.

Furthermore, the compounds of the formula (I) according to the present invention are suitable for suppressing the growth of tumour cells in humans and mammals. This is based on an interaction of the compounds according to the present invention with tubulin and microtubules and through encouragement of microtubule polymerization.

For this purpose, an effective quantity of one or more compounds of the formula (I) or pharmaceutically compatible salts thereof may be administered.

The active ingredients may be applied as such, in the form of their formulations or the application forms prepared therefrom, such as ready-to-use solutions, suspensions, spray powders, pastes, soluble powders, dusting agents, and granules. The application is performed in the typical way, e.g., through pouring, spraying, scattering, dusting, foaming, painting, etc. Furthermore, it is possible to apply the active ingredients according to the ultralow volume method or inject the active ingredient preparation or the active ingredient itself into the soil. The seed of the plants may also be treated.

When using the active ingredients according to the present invention as fungicides, the applied quantities may be varied within a wide range depending on the type of application. When treating plant parts, the applied quantities of active ingredient are generally between 0.1 and 10,000 g/hectare, preferably between 10 and 1000 g/hectare. When seeds are treated, the applied quantities of active ingredient are generally between 0.001 and 50 g per kilogram of seed, preferably between 0.01 and 10 g per kilogram of seed. When treating the soil, the applied quantities of active ingredient are generally between 0.1 and 10,000 g/hectare, preferably between 1 and 5000 g/hectare.

As already noted above, all plants and their parts may be treated according to the present invention. In a preferred embodiment, types of plants and plant species occurring wild or obtained through conventional biological cultivation methods, such as breeding or protoplast fusion, as well as their parts, may be treated. In a further preferred embodiment, transgenic plants and plant species which were obtained through methods of genetic engineering, optionally in combination with conventional methods (genetically modified organisms) and their parts are treated. The term “parts” and/or “parts of plants” or “plant parts” was explained above.

According to the present invention, plants of the particular commercially available plant species or plant species in use are especially preferably treated. Plant species are understood as plants having new properties (“traits”), which may be cultivated both through conventional cultivation, through mutagenesis, or through recombinant DNA technologies. These may be species, breeds, biotypes, and genotypes.

Depending on the plant types and/or plant species, their location and growth conditions (soil, climate, vegetation period, nutrition), synergistic effects may also arise through the treatment according to the present invention. Thus, for example, lowered applied quantities and/or expansions of the activity spectrum and/or an amplification of the effect of the materials and agents usable according to the present invention, better plant growth, elevated tolerance to high or low temperatures, elevated tolerance to drought or to water and/or soil salinity, elevated blooming performance, easier harvesting, acceleration of ripening, higher harvest yields, higher quality and/or higher nutritional value of the harvested products, greater storage capability and/or processability of the harvested products are possible, which exceed the actual effects to be expected.

The preferred transgenic (obtained through genetic engineering) plants and/or plant species to be treated according to the present invention include all plants which have obtained genetic material through genetic modification which provides these plants with especially advantageous valuable properties (“traits”). Examples of such properties are better plant growth, elevated tolerance to high or low temperatures, elevated tolerance to drought or to water and/or soil salinity, elevated blooming performance, easier harvesting, acceleration of ripeness, elevated harvest yields, greater storage capability and/or processability of the harvested products. Further and especially pronounced examples of such properties are elevated defense of the plants against animal and microbial pests, for example, against insects, mites, phytopathogenic fungi, bacteria, and/or viruses, as well as elevated tolerance of the plants to specific herbicidal active ingredients. Examples of transgenic plants include the important cultured plants, such as grains (wheat, rice), maize, soya, potatoes, cotton, tobacco, rapeseed, as well as fruit plants (having the fruits apples, pears, citrus fruits, and grapes), with maize, soya, potatoes, cotton, tobacco, and rapeseed being noted in particular. The elevated defense of the plants to insects, arachnids, nematodes, and snails through toxins arising in the plants, particularly those which are generated in the plants by the genetic material of Bacillus thuringiensis (e.g., for example, by the genes CryIA(a), CryIA(b), CryIA(c), CryIIA, CrylIIA, CryIIIB2, Cry9c Cry2Ab, Cry3Bb and CryI, as well as their combinations) are especially to be noted (referred to in the following as “Bt plants”). The elevated defenses of plants against fungi, bacteria, and viruses through systemic acquired resistance (SAR), systemin, phytoalexines, elicitors, and resistance genes and correspondingly expressed proteins and toxins are also especially noted as properties (“traits”). The elevated tolerance of the plants to specific herbicidal active ingredients, such as imidazolinones, sulphonyl ureas, glyphosates, or phosphinotricine (e.g., “PAT” gene) is also especially to be noted. The particular genes which provide the desired properties (“traits”) may also occur in the transgenic plants in combination with one another. Examples of “Bt plants” are maize varieties, cotton varieties, soya varieties, and potato varieties which are distributed under the trade names YIELD GARD® (e.g., maize, cotton, soya), KnockOut® (e.g., maize), StarLink® (e.g., maize), Boligard® (cotton), Nucoton® (cotton) and NewLeaf® (potato). Examples of plants tolerant to herbicides are maize varieties, cotton varieties and soya varieties, which are distributed under the trade names Roundup Ready® (tolerance to glyphosates, e.g., maize, cotton, soya), Liberty Link® (tolerance to phosphinotricine, e.g., rapeseed), IMI® (tolerance to imidazolinones), and STS® (tolerance to sulphonyl ureas, e.g., maize). The varieties (e.g., maize) of plants resistant to herbicides (conventionally cultivated for herbicide tolerance) distributed under the trade name Clearfield® are also noted. Of course, the statements also apply for plant varieties developed in the future and/or coming to market in the future having these genetic properties (“traits”) or those developed in the future.

The plants listed may be treated especially advantageously according to the present invention using the compounds of the general formula (I) and/or the active ingredient mixtures according to the present invention. The preferred ranges specified above for the active ingredients and/or mixtures also apply for the treatment of these plants. The plant treatment using the compounds and/or mixtures specially listed in the present text is especially noted.

The production and the use of the active ingredients according to the present invention is described in the following examples.

### PRODUCTION EXAMPLES

### Example 1

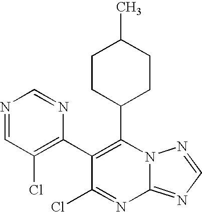 Preparation of Grignard Solution:

To prepare a Grignard solution, a solution of 5.0 g (28.235 mmol) 4-methyl-cyclohexylbromide in 25 ml diethylether is dripped into a mixture made of 0.686 g (28.235 mmol) magnesium shavings and 15 ml diethylether at room temperature under argon atmosphere. After brief heating, the exothermic reaction begins. The reaction mixture is stirred until the magnesium shavings have completely dissolved. In this way, a 0.7 molar Grignard solution of 4-methyl-cyclohexyl magnesium bromide in diethylether is obtained, which is used in the freshly produced state for further synthesis.

Method (a)

2.9 ml of the previously produced 0.7 molar solution of 4-methyl-cyclohexyl magnesium bromide in diethylether (Grignard solution) is dripped into a solution of 0.51 g (1.69 mmol) 5,7-dichloro-6-(5-chloro-4-pyrimidinyl)[1,2,4]triazolo[1,5-a]pyrimidine in 15 ml tetrahydrofuran, 1.5 ml N-methylpyrrolidone and 28 mg iron(III) acetonylacetonate at room temperature under argon atmosphere. The mixture is stirred for 2 hours at room temperature, a further 1 ml of the Grignard solution is added, and the mixture is stirred for a further hour. The reaction mixture is then admixed with 10 ml acetic ethyl ester and 1 ml 1 N aqueous hydrochloric acid and stirred 5 minutes at room temperature.

The organic phase is separated and the aqueous phase is extracted with a further 10 ml acetic ethyl ester. The combined organic phases are dried over sodium sulphate and concentrated under reduced pressure. The residue is filtered via a short column of silica gel using cyclohexane/acetic ethyl ester (3:1).

77 mg (12% of theoretical yield) of 5-chloro-6-(5-chloro-4-pyrimidinyl)-7-(4-methylcyclohexyl)[1,2,4]triazolo[1,5-a]pyrimidine is obtained.

HPLC: logP=3.27

### Example 2

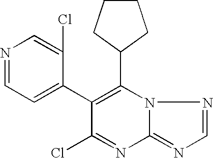 Method (a)

1 ml of a previously produced 2 molar solution of cyclopentylmagnesium bromide in diethylether (Grignard solution) is dripped into a solution of 0.50 g (1.66 mmol) 5,7-dichloro-6-(5-chloro-4-pyrimidinyl)[1,2,4]triazolo[1,5-a]-pyrimidine and 28 mg iron (III)acetonylacetonate in 1.5 ml tetrahydrofuran and 1,5 ml N-methylpyrrolidone at room temperature under argon atmosphere. The mixture is stirred 2 hours at room temperature and the reaction mixture is then admixed with 10 ml acetic ethyl ester and 1 ml 1 N aqueous hydrochloric acid. The organic phase is separated, dried over sodium sulphate, and concentrated under reduced pressure. 73 mg (11.6% of theoretical yield) of 5-chloro-6-(5-chloro-4-pyrimidinyl)-7-cyclopentyl[1,2,4]triazolo[1,5-a]pyrimidine is obtained.

HPLC: LogP=2.50

The compounds of the formula (I) listed in the following Table 1 are also obtained according to the methods specified above.

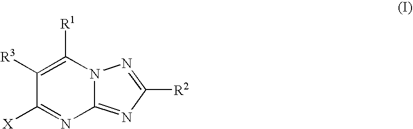

TABLE 1

| Ex. | | | | | | |
|---|---|---|---|---|---|---|
| No. | R¹ | R² | R³ | X | logP* | f.p. (° C.) |
| 3 | 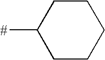 | H | 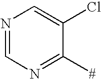 | Cl | 2.79 | |
| 4 | 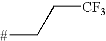 | H | 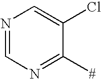 | Cl | 2.21 | |
| 5 | 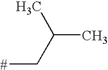 | H | 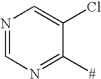 | Cl | 2.26 | |
| 6 | 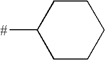 | H | 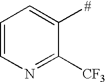 | Cl | | |
| 7 | 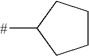 | H | 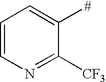 | Cl | | |
| 8 | 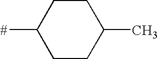 | H | 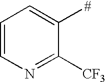 | Cl | | |
| 9 | 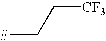 | H | 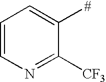 | Cl | | |
| 10 | 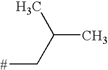 | H | 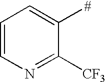 | Cl | | |

# represents the linkage point
*) The logP values were determined in accordance with EEC directive 78/831 Annex V. A8 through HPLC (gradient method, acetonitrile/0.1% aqueous phosphoric acid).

Production of Precursor Products of the Formula (II)

### Example 11

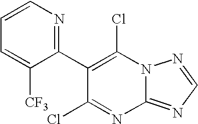 Method (b)

8 g (16 mmol) 6-(3-trifluoromethyl-pyridin-2-yl)-[1,2,4]triazolo[1,5-a]pyrimidin-5,7-diol is stirred with 12 ml phosphorus oxychloride. 2.7 g phosphorus pentachloride is then added in portions. The mixture is heated 2 hours under reflux. After cooling, the reaction mixture is concentrated under reduced pressure, admixed with 100 ml water, and extracted 3 times using 100 ml dichloromethane each time. The combined organic phases are washed 2 times using 50 ml of water, dried over sodium sulphate, and concentrated under reduced pressure. The residue is chromatographed using dichloromethane/methyl-t-butylether (95:5) on silica gel. 1.4 g (25.7% of theoretical yield) of 5,7-dichloro-6-(3-trifluormethyl-pyridin-2-yl)-[1,2,4]triazolo[1,5-a]pyrimidine is obtained.

HPLC: logP=1.97

### Example 12

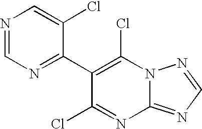 Method (b)

8 g (16 mmol) 6-(5-chloro-4-pyrimidinyl)[1,2,4]triazolo[1,5-a]pyrimidin-5,7-diol is stirred with 25 ml phosphorus oxychloride. 3.1 g phosphorus pentachloride is added in portions. The mixture is stirred 3 hours at 110° C. After cooling to room temperature, the reaction mixture is admixed with 300 ml water and extracted three times using 100 ml dichloromethane each time. The combined organic phases are dried over sodium sulphate and concentrated under reduced pressure. The residue is chromatographed using hexane/acetic ethyl ester (9:1-5:1) on silica gel. 1.4 g (25.7% of theoretical yield) of 5,7-dichloro-6-(5-chloro-4-pyrimidinyl)[1,2,4]triazolo[1,5-a]pyrimidine is obtained.

HPLC: logP=1.43

### Example 13

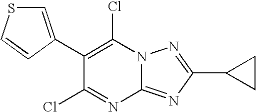

A mixture made of 2.0 g (10.74 mmol) 2-thienyl malonic acid and 1.33 g (10.74 mmol) 3-amino-5-cyclo-propyl-1,2,4-triazol is admixed at room temperature within 2 minutes with 41.13 g (286 mmol) phosphorus oxychloride while stirring. The mixture is then heated to 90° C. for 18 hours and then cooled to room temperature. The reaction mixture is poured into 250 ml ice water, and the resulting suspension is stirred 1 hour. The mixture is suctioned off and washed using 50 ml water. For further purification, the product is suspended in 50 ml cyclohexane/acetic ethyl ester=1:1 and boiled briefly, then cooled, suctioned via a short silica gel column, and washed 8 times using 50 ml cyclohexane/acetic ethyl ester=1:1 each time. The filtrate is dried over sodium sulphate and then filtered again. The filter residue is washed down using a little cyclohexane/acetic ethyl ester=1:1. The entire filtrate is concentrated under reduced pressure. 1.73 g (50.7% of theoretical yield) of 5,7-dichloro-2-cyclopropyl-6-(thien-3-yl)-[1,2,4]triazolo[1,5-a]pyrimidine is obtained in the form of a beige solid.

### Example 14

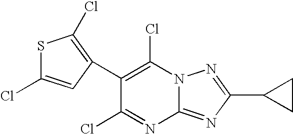

A chlorine gas stream is introduced into a solution of 6.0 g (19,28 mmol) 5,7-dichloro-2-cyclopropyl-6-(thien-3-yl).[1,2,4]triazolo[1,5-a]pyrimidine in 80 ml acetic acid for 2 hours at room temperature. The reaction mixture is then concentrated under reduced pressure. The remaining residue is chromatographed using cyclohexane/acetic ethyl ester=2:1 on silica gel. The residue obtained after concentrating the eluate is stirred with cyclohexane/acetic acid=1:1, then suctioned and dried. The previously obtained mother liquor is chromatographed using cyclohexane/acetic ethyl ester=1:1 on silica gel after concentration. 2.7 g (50.5% of theoretical yield) of 5,7-dichloro-2-cyclopropyl-6-(2,5-dichloro-thien-3-yl)-[1,2,4]triazolo[1,5-a]pyrimidine is obtained in this way.

### Example 15

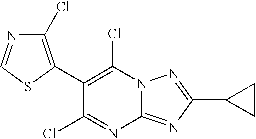

A solution of 17.0 g (54.89 mmol) 2-cyclopropyl-6-(4-chloro-thiazol-5-yl)-[1,2,4]triazolo[1,5-a]pyrimidin-5,7-diol in 51.2 ml phosphorus oxychloride is admixed in portions with 5.72 g (27.44 mmol) phosphorus pentachloride at room temperature while stirring. After admixing, the reaction mixture is stirred for 3 hours at 110° C., then cooled to room temperature and poured onto ice water. The mixture is extracted multiple times using dichloromethane, the organic phase is dried over sodium sulphate and concentrated under reduced pressure. The remaining residue is chromatographed using cyclohexane/acetic ethyl ester=3:1 on silica gel. 0.35 g (1.66% of theoretical yield) of 5,7-dichloro-2-cyclopropyl-6-(4-chloro-thiazol-5-yl)-[1,2,4]-triazolo[1,5-a]pyrimidine is obtained in this way.

HPLC: logP=2.46

Production of Precursor Products of the Formula (VI)

### Example 16

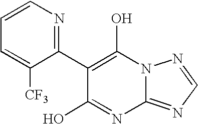 Method (c)

5.5 g (19.84 mmol) 2-(3-trifluoromethyl-pyridin-2-yl)-malonic dimethylester and 1.67 g (19.84 mmol) 3-amino-1,2,4-triazole are stirred in 5,2 ml tributylamine for 2 hours at 180° C. The methanol resulting during the reaction is distilled off continuously. After cooling, the desire product separates from the tributylamine. The tributylamine is decanted off and the 6-(3-trifluoromethyl-pyridin-2-yl)-[1,2,4]triazolo[1,5-a]pyrimidin-5,7-diol obtained (yield: approximately 8 g, 60% purity) is used without further purification in the next reaction step.

HPLC: logP=−0.23

### Example 17

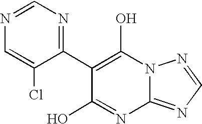 Method (c)

10 g (40.9 mmol) 2-(5-chloro-pyrimidin-4-yl)-malonic dimethylester and 3.44 g (40.9 mmol) 3-amino-1,2,4-triazole are stirred in 10.7 ml tributylamine for 2 hours at 185° C. The methanol resulting during the reaction is distilled off continuously. After cooling, the desire product separates from the tributylamine. The tributylamine is decanted off and the 6-(5-chloro-4-pyrimidinyl)[1,2,4]triazolo[1,5-a]pyrimidin-5,7-diol obtained (yield: approximately 15 g, 11% purity, approximately 15% of theoretical yield) is used without further purification in the next reaction step.

HPLC: logP=−0.23

### Example 18

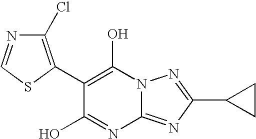

A mixture made of 8.5 g (34.05 mmol) 2-(4-chloro-thiazol-5-yl)malonic dimethylester, 4.23 g (34.05 mmol) 3-amino-5-cyclopropyl-1,2,4-triazole, and 8,92 ml tri-n-butylamine is stirred at 185° C. for 2 hours. At the same time, the methanol resulting from the reaction is distilled off. The mixture is cooled to room temperature and the separating tri-n-butylamine is decanted off. 18 g of a product, which, according to HPLC, includes 64% 2-cyclopropyl-6-(4-chloro-thiazol-5-yl)-[1,2,4]triazolo[1,5-a]pyrimidin-5,7-diol is obtained in this way.

HPLC: logP=0.10

Production of Precursor Products of the Formula (VII-a)

### Example 19

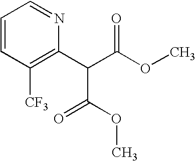 Method (d)

9 g (207 mmol) 60% sodium hydride suspension is suspended in 300 ml dioxane. 27.29 g (206.6 mmol) malonic dimethylester is dripped into this mixture at 55-60° C. and stirred for a further 30 minutes at the same temperature. After adding 8.18 g (82.63 mmol) copper(I) chloride the mixture is heated to 80° C. and then 15 g (82.63 mmol) 2-chloro-3-trifluormethylpyridine is dripped in. The reaction mixture is now stirred 14 hours at 100° C. After the subsequent cooling to 15-20° C., concentrated hydrochloric acid is dripped in slowly until the mixture is acidic. 600 ml water and 300 ml dichloromethane are now added and insoluble components are filtered off. The organic phase is separated from the filtrate, dried over sodium sulphate, and concentrated under reduced pressure. The residue is chromatographed using hexane/acetic ester (4:1) on silica gel. 10.1 g (40% of theoretical yield) of 2-[3-trifluoromethyl]-pyrimidin-2-yl)malonic dimethylester is obtained.

HPLC: logP=2.05

Production of Precursor Products of the Formula (VII-b)

### Example 20

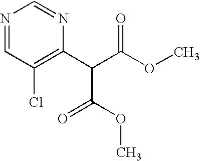 Method (e)

2.6 g (65.4 mmol) 60% sodium hydride suspension is suspended in 100 ml tetrahydrofuran. 6.9 g (52.4 mmol) malonic dimethylester is added at 0° C. and the mixture is stirred for 0.5 hours at the same temperature. A solution of 6.5 g (43.63 mmol) 4,5-dichloropyrimidine in 50 ml tetrahydrofuran is then dripped in and the mixture is stirred a further 3 hours at room temperature. 150 ml 1 N hydrochloric acid is then slowly dripped in and the mixture is then extracted using 100 ml dichloromethane. The organic phase is separated off, dried over sodium sulphate, and concentrated under reduced pressure. The residue is chromatographed on silica gel using methyl-t-butylether/petroleum ether (1:9). 7 g (65.6% of theoretical yield) of 2-(5-chloro-4-pyrimidin-2-yl)malonic dimethylester is obtained.

HPLC: logP=1.33

Production of 4,5-dichloropyrimidine

### Example 21

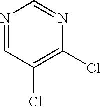

1.6 ml dimethylamine is added to a solution of 112.5 g (673,7 mmol) 5-chloro-6-oxo-1,6-dihydropyrimidin-1-ium chloride in 630 ml phosphorus oxychloride and heated for 3 hours under reflux. The excess phosphorus oxychloride is then distilled off under reduced pressure. After cooling, the residue is poured onto 1.5 l icewater, extracted using 500 ml dichloromethane, the organic phase is dried over sodium sulphate and concentrated under reduced pressure. 72.3 g (66.3% of theoretical yield) 4,5-dichloropyrimidine is obtained.

HPLC: logP=1.35

Production of 5-chloro-6-oxo-1,6-dihydropyrimidin-1-ium chloride

### Example 22

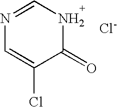

6.5 g (40 mmol) iron(III) chloride is added to a solution of 77 g (0.8 mol) 4(3H)-pyrimidinone in 770 ml glacial acetic acid and 113.6 g (1.6 mol) chlorine is introduced within 2 hours at 40-45° C. The reaction mixture is cooled to 15° C., the resulting solid product is suctioned off and washed using ether. 112.5 g (84% of theoretical yield) 5-chloro-6-oxo-1,6-dihydropyrimidin-1-ium chloride is obtained.

Production of 4(3H)-pyrimidinone

### Example 23

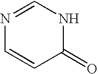

A mixture of 103 g (0.804 mol) 6-mercapto-4(1H)-pyrimidinone (JP 50053381, Chem. Abstr. CAN 84:17404) and 141.5 g (1.2 Mol) Raney nickel in 1.2 l ethanol is heated for 8 hours under reflux. The solution is filtered hot, the residue is washed with ethanol, and the filtrate is concentrated under reduced pressure. 67.2 g (87% of theoretical yield) 4(3H)-pyrimidinone is obtained.

### USAGE EXAMPLES

### Example A

Podosphaera Test (Apple)/Protective

Solvent: 24.5 parts by weight acetone

24.5 parts by weight dimethylacetamide
   Emulsifier: 1 part by weight alkyl-aryl polyglycolether

To produce an effective active ingredient preparation, 1 part by weight active ingredient is mixed with the specified quantities of solvent and emulsifier and the concentrate is diluted using water to the desired concentration.

To test for protective activity, young plants are sprayed with the active ingredient preparation in the specified applied quantity. After drying of the spray coating, the plants are inoculated with an aqueous spore suspension of the apple powdery mildew pathogen Podosphaera ieucotricha. The plants are then placed in a greenhouse at approximately 23° C. and a relative ambient humidity of approximately 70%.

The analysis is performed 10 days after the inoculation. In this case, 0% means an activity which corresponds to that of the control, while an activity of 100% means that no infection is observed.

In this test, the materials according to the present invention listed in Examples 1, 2, 3, and 4 display an activity of over 85% at an applied quantity of 100 g/ha.

### Example B

Venturia Test (Apple)/Protective

Solvent: 24.5 parts by weight acetone

24.5 parts by weight dimethylacetamide
   Emulsifier: 1 part by weight alkyl-aryl polyglycolether

To produce an effective active ingredient preparation, 1 part by weight active ingredient is mixed with the specified quantities of solvent and emulsifier and the concentrate is diluted using water to the desired concentration.

To test for protective activity, young plants are sprayed with the active ingredient preparation in the specified applied quantity. After drying of the spray coating, the plants are inoculated with an aqueous conidia suspension of the apple scab pathogen Venturia inaequalis and then remain 1 day in an incubation chamber at approximately 20° C. and a relative ambient humidity of approximately 100%.

The plants are then placed in a greenhouse at approximately 21° C. and a relative ambient humidity of approximately 90%.

The analysis is performed 10 days after the inoculation. In this case, 0% means an activity which corresponds to that of the control, while an activity of 100% means that no infection is observed.

In this test, the materials according to the present invention listed in Examples 1, 2, 3, and 4 display an activity of 90% or more at an applied quantity of 100 g/ha.

### Example C

Alternaria Test (Tomato)/Protective

Solvent: 49 parts by weight N,N-dimethyl formamide

Emulsifier: 1 part by weight alkyl aryl polyglycolether

To produce an effective active ingredient preparation, 1 part by weight active ingredient is mixed with the specified quantities of solvent and emulsifier and the concentrate is diluted using water to the desired concentration.

To test for protective activity, young tomato plants are sprayed with the active ingredient preparation in the specified applied quantity. 1 day after the treatment, the plants are inoculated with a spore suspension of Alternaria solani and then stand 24 hours at 100% relative humidity and 20° C. The plants are then stand at 96% relative humidity and a temperature of 20° C.

The analysis is performed 7 days after the inoculation. In this case, 0% means an activity which corresponds to that of the control, while an activity of 100% means that no infection is observed.

In this test, the materials according to the present invention listed in Examples 1, 2 and 3 display an activity of 100% at an applied quantity of 750 g/ha.

1. A compound of the formula 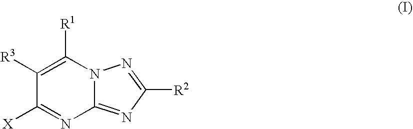
in which
R¹ represents optionally substituted alkyl, optionally substituted alkenyl, optionally substituted alkynyl, optionally substituted cycloalkyl, optionally substituted cycloalkenyl, or optionally substituted heterocyclyl, which is linked via carbon,
R² represents hydrogen, halogen, optionally substituted alkyl, or optionally substituted cycloalkyl,
R³ represents optionally substituted heterocyclyl,
X represents halogen, cyano, optionally substituted alkyl, optionally substituted alkoxy, optionally substituted alkylthio, optionally substituted alkylsulphinyl, or optionally substituted alkylsulphonyl.

2. The compound of claim 1, in which
R¹ represents alkyl having 1 to 6 carbon atoms, which may be substituted one to five times, identically or differently, by halogen, cyano, hydroxy, alkoxy having 1 to 4 carbon atoms, tri(C₁-C₄ alkyl)silyl or cycloalkyl having 3 to 6 carbon atoms, which may be substituted one to three times, identically or differently by halogen, halogenalkyl having 1 or 2 carbon atoms and 1 to 5 halogen atoms or alkyl having 1 to 4 carbon atoms, or
R¹ represents alkenyl having 2 to 6 carbon atoms, which may be substituted one to three times, identically or differently by halogen, cyano, hydroxy, alkoxy having 1 to 4 carbon atoms, tri(C₁-C₄ alkyl)silyl or cycloalkyl having 3 to 6 carbon atoms, which may be substituted one to three times, identically or differently by halogen, halogenalkyl having 1 or 2 carbon atoms and 1 to 5 halogen atoms or alkyl having 1 to 4 carbon atoms, or
R¹ represents alkynyl having 3 to 6 carbon atoms, which may be substituted one to three times, identically or differently by halogen, cyano, alkoxy having 1 to 4 carbon atoms, tri(C₁-C₄ alkyl)silyl or cycloalkyl having 3 to 6 carbon atoms, which may be substituted one to three times, identically or differently by halogen, halogenalkyl having 1 or 2 carbon atoms and 1 to 5 halogen atoms or alkyl having 1 to 4 carbon atoms, or
R¹ represents cycloalkyl having 3 to 6 carbon atoms, which may be substituted one to three times, identically or differently by halogen, halogenalkyl having 1 or 2 carbon atoms and 1 to 5 halogen atoms or alkyl having 1 to 4 carbon atoms, or
R¹ represents cycloalkenyl having 3 to 6 carbon atoms, which may be substituted one to three times, identically or differently by halogen or alkyl having 1 to 4 carbon atoms, or
R¹ represents saturated or unsaturated heterocyclyl, linked via carbon, having 5 or 6 ring members and 1 to 3 heteroatoms, such as nitrogen, oxygen, or sulphur, the heterocyclyl optionally substituted once or twice by halogen, alkyl having 1 to 4 carbon atoms, cyano, nitro, alkoxy having 1 to 4 carbon atoms, cycloalkyl having 3 to 6 carbon atoms, halogenalkyl having 1 to 4 carbon atoms and 1 to 9 halogen atoms, or halogenalkoxy having 1 to 4 carbon atoms and 1 to 9 halogen atoms
R² represents hydrogen, fluorine, chlorine, bromine, iodide, alkyl having 1 to 4 carbon atoms, halogenalkyl having 1 to 4 carbon atoms and 1 to 9 halogen atoms, or cycloalkyl having 3 to 6 carbon atoms,
R³ represents saturated or unsaturated heterocyclyl having 5 or 6 ring members and 1 to 4 heteroatoms, such as oxygen, nitrogen or sulphur, the heterocyclyl optionally substituted one to four times, identically or differently by fluorine, chlorine, bromine, cyano, nitro, alkyl, alkoxy, hydroximinoalkyl or alkoximinoalkyl each having 1 to 3 carbon atoms per alkyl part,
halogenalkyl or halogenalkoxy each having 1 to 3 carbon atoms and 1 to 7 halogen atoms,
and
X represents fluorine, chlorine, bromine, cyano, alkyl having 1 to 4 carbon atoms, alkoxy having 1 to 4 carbon atoms, alkylthio having 1 to 4 carbon atoms, alkylsulphinyl having 1 to 4 carbon atoms, or alkylsulphonyl having 1 to 4 carbon atoms.

3. The compound of claim 1 or 2, in which
R¹ represents a residue of the formula 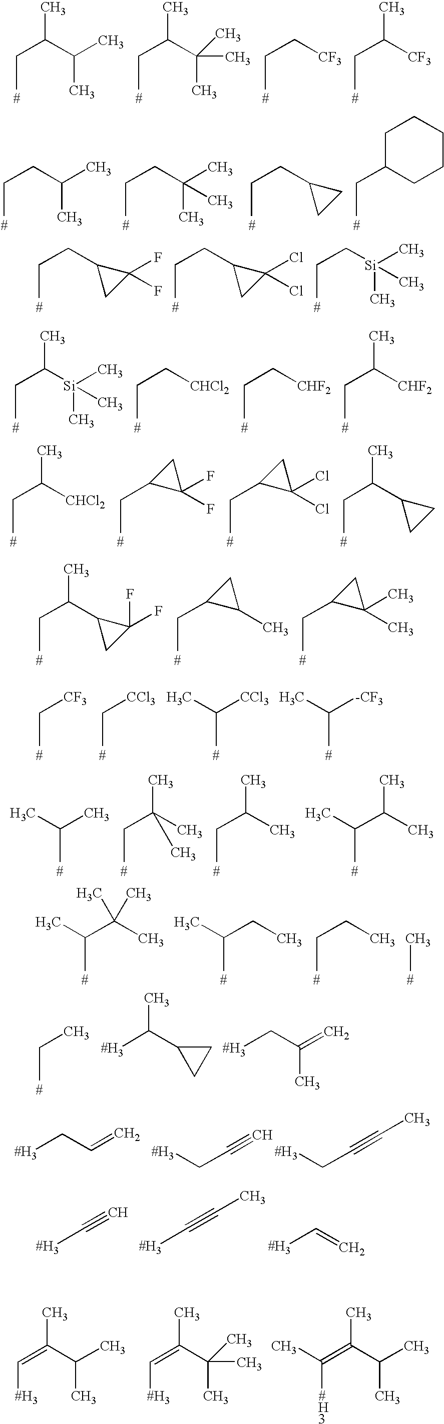 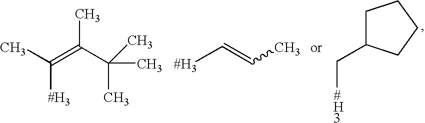
or
R¹ represents a residue of the formula 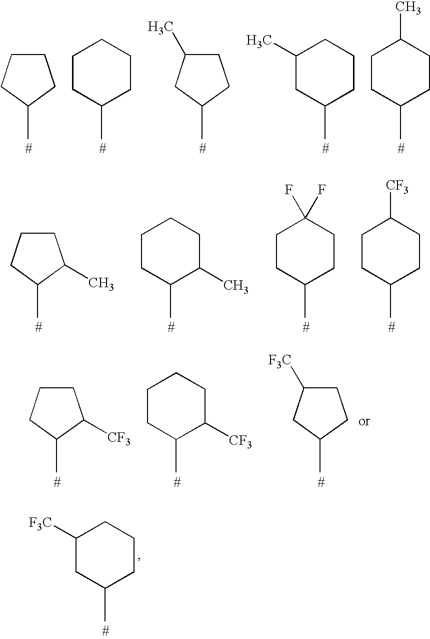
or
R¹ represents a residue of the formula 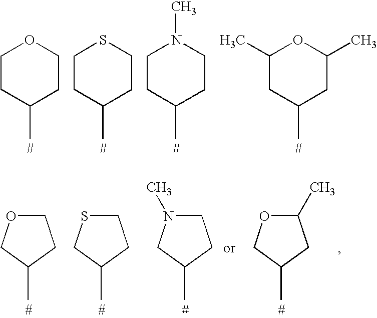
wherein # marking marks the linkage point in each case,
R² represents hydrogen, fluorine, chlorine, bromine, iodide, methyl, ethyl, isopropyl, cyclopropyl, cyclobutyl, cyclopentyl, cyclohexyl, trifluoromethyl, 1-trifluoromethyl-2,2,2-trifluorethyl, or heptafluoroisopropyl,
R³ represents pyridyl, which is linked in the second or fourth position and may be substituted one to four times, identically or differently, by fluorine, chlorine, bromine, cyano, nitro, methyl, ethyl, methoxy, methylthio, hydroximinomethyl, hydroximinoethyl, methoximinomethyl, methoximinoethyl or trifluoromethyl, or
R³ represents pyrimidyl, which is linked in the second or fourth position and may be substituted one to three times, identically or differently, by fluorine, chlorine, bromine, cyano, nitro, methyl, ethyl, methoxy, methylthio, hydroximinomethyl, hydroximinoethyl, methoximinomethyl, methoximinoethyl or trifluoromethyl, or
R³ represents thienyl, which is linked in the second or third position and may be substituted one to three times, identically or differently, by fluorine, chlorine, bromine, cyano, nitro, methyl, ethyl, methoxy, methylthio, hydroximinomethyl, hydroximinoethyl, methoximinomethyl, methoximinoethyl or trifluoromethyl, or
R³ represents thiazolyl, which is linked in the second, fourth, or fifth position and may be substituted once or twice, identically or differently, by fluorine, chlorine, bromine, cyano, nitro, methyl, ethyl, methoxy, methylthio, hydroximinomethyl, hydroximinoethyl, methoximinomethyl, methoximinoethyl or trifluoromethyl,
and
X represents fluorine, chlorine, bromine, cyano, methyl, methoxy, or methylthio.

4. A method of preparing a compound of claim 1 comprising:
(a) contacting dihalogen triazolopyrimidines of the formula 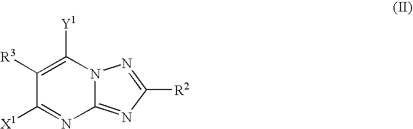
in which
R², R³, and R⁴ have the meanings specified in claim 1,
X¹ represents halogen and
Y¹ represents halogen
with metal compounds of the formula (III),
R¹-Me (III)
in which
R¹ has the meanings specified in claim 1,
Me represents lithium, dihydroxyboranyl or a residue of the formula 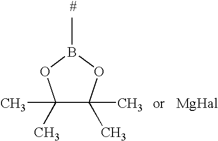
wherein # marks the linkage point,
in which
Hal represents chlorine or bromine,
optionally in the presence of a diluent,
optionally in the presence of an acid acceptor,
and
optionally in the presence of a catalyst and the triazolopyrimidines of the formula (Ia) thus obtained are optionally reacted 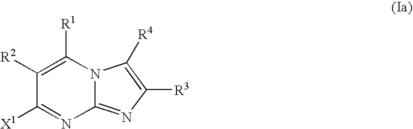
in which
R¹, R², R³ and X¹ have the meanings specified in claim through 1,
either
α) with compounds of the formula
R⁴-Me¹ (IV)
in which
R⁴ represents optionally substituted alkoxy, optionally substituted alkylthio, optionally substituted alkylsulphinyl, optionally substituted alkylsulphonyl, or cyano
Me¹ represents sodium or potassium,
optionally in the presence of a diluent,
or
β) with Grignard compounds of the formula
R⁵—MgHal¹ (V)
in which
R⁵ represents optionally substituted alkyl and
Hal¹ represents chlorine or bromine,
in the presence of a diluent.

5-8. (canceled)

9. A compound of the formula 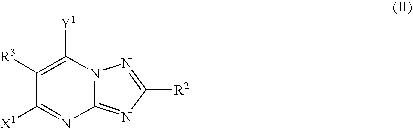
in which
R² represents hydrogen, halogen, optionally substituted alkyl or optionally substituted cycloalkyl,
R³ represents optionally substituted heterocyclyl,
X¹ represents halogen and
Y¹ represents halogen.

10. A method for preparing the compound of claim 9 comprising:
contacting a compound of the formula 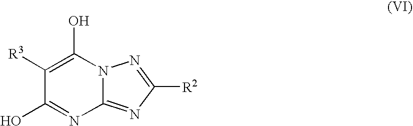
in which
R² and R³ which have the meanings specified in claim 9,
with halogenation agents, optionally in the presence of a diluent,
wherein a compound of claim 9 is prepared.

11. The compound of claim 10 of the formula 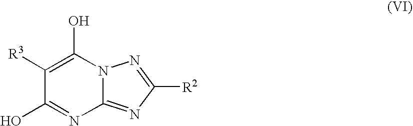
in which
R² represents hydrogen, halogen, optionally substituted alkyl or optionally substituted cycloalkyl,
R³ represents optionally substituted cycloalkyl.

12. A method for preparing the compound of claim 11, comprising:
contacting a compound of the formula 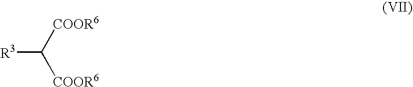
in which
R³ has the meaning specified in claim 11 and
R⁶ represents alkyl having 1 to 4 carbon atoms,
with a compound of the formula 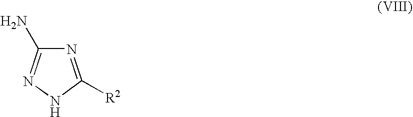
in which
R² has the meaning specified in claim 11,
optionally in the presence of a diluent and optionally in the presence of an acid binder, wherein a compound of claim 11 is prepared.

13. A compound of the formula 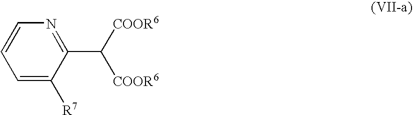
in which
R⁶ represents alkyl having 1 to 4 carbon atoms and
R⁷ represents halogen or halogenalkyl.

14. A method for preparing the compound of claim 13 comprising:
contacting a compound of the formula 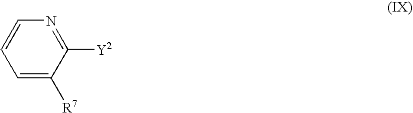
in which
R⁷ has the meaning specified in claim 13 and
Y² represents halogen,
with malonic esters of the formula 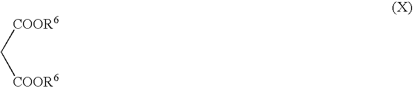
in which
R⁶ has the meaning specified in claim 13,
optionally in the presence of a diluent, optionally in the presence of a copper salt, and optionally in the presence of an acid acceptor, wherein a compound of claim 13 is prepared.

15. A compound of the formula 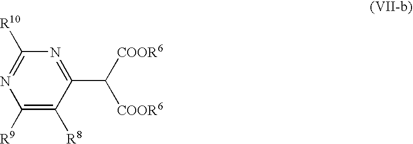
in which
R⁶ represents alkyl having 1 to 4 carbon atoms,
R⁸ represents halogen or halogenalkyl, and
R⁹ and R¹⁰ independently of one another, represent hydrogen, fluorine, chlorine, bromine, methyl, ethyl or methoxy.

16. A method for preparing the compound of claim 15, comprising:
contacting a compound of the formula 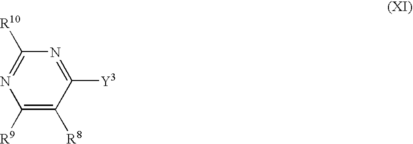
in which
R⁸, R⁹ and R¹⁰ have the meanings specified in claim 15 and
Y³ represents halogen,
with malonic esters of the formula 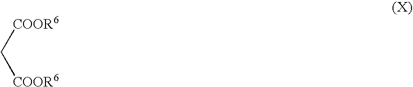
in which
R⁶ has the meaning specified in claim 15,
optionally in the presence of a diluent, optionally in the presence of a copper salt, and optionally in the presence of an acid acceptor, wherein a compound of claim 15 is prepared.

17. A composition comprising at least one compound of claim 1.

18. The composition of claim 17, further comprising extenders and/or surfactants.

19. A method for combating undesired micro-organisms, comprising contacting said micro-organisms or said micro-organisms' living space or both with the composition of claim 17.

20. A method for preparing the composition of claim 18, comprising mixing one or more said compounds with said extenders and/or surfactants.
